(19) United States
(12) Patent Application Publication
(10) Pub. No.: US 2024/0300820 A1
(43) Pub. Date: Sep. 12, 2024

(54) EMM-74 MOLECULAR SIEVE COMPOSITIONS, SYNTHESES, AND USES

(71) Applicant: ExxonMobil Technology and Engineering Company, Annandale, NJ (US)

(72) Inventors: Trong D. Pham, Flemington, NJ (US); Eugene A. Terefenko, Center Valley, PA (US); Mary Abdulkarim, Palisades Park, NJ (US)

(21) Appl. No.: 18/593,677

(22) Filed: Mar. 1, 2024

Related U.S. Application Data
(60) Provisional application No. 63/488,912, filed on Mar. 7, 2023.

(51) Int. Cl. C01B 39/48; B01J 29/70; B01J 29/86; B01J 35/63; B01J 37/00; B01J 37/03; B01J 37/04; C01B 37/00; C01B 39/02; C07D 471/04

(52) U.S. Cl. CPC C01B 39/48; B01J 29/70; B01J 29/86; B01J 35/633; B01J 37/0018; B01J 37/031; B01J 37/04; C01B 37/007; C01B 39/026; C07D 471/04; C01P 2002/72; C01P 2004/03; C01P 2004/12; C01P 2004/20; C01P 2006/12; C01P 2006/14

(57) ABSTRACT

Molecular sieves, designated as EMM-74, characterized by a unique powder XRD pattern, methods of making the same, and uses thereof.

### CROSS REFERENCE TO RELATED APPLICATIONS

The present application claims priority to and the benefit of U.S. Provisional Application No. 63/488,912 filed on Mar. 7, 2023, which is hereby incorporated by reference in its entirety.

### TECHNICAL FIELD

The present disclosure relates to molecular sieve compositions, methods of making the same, and uses thereof.

### BACKGROUND OF THE INVENTION

Molecular sieve materials, both natural and synthetic, may be used as adsorbents and have catalytic properties for hydrocarbon conversion reactions. Certain molecular sieves, such as zeolites, AlPOs, and mesoporous materials, are ordered, porous crystalline materials having a definite crystalline structure as determined by X-ray diffraction (XRD). Certain molecular sieves are ordered and produce specific identifiable XRD patterns. Within certain molecular sieve materials there may be a large number of cavities, which may be interconnected by a number of channels or pores. These cavities and pores are uniform in size within a specific molecular sieve material. Because the dimensions of these pores are such as to accept for adsorption molecules of certain dimensions while rejecting those of larger dimensions, these materials have come to be known as “molecular sieves” and are utilized in a variety of industrial processes, e.g., cracking, hydrocracking, disproportionation, alkylation, oligomerization, and isomerization.

Molecular sieves that find application in catalysis and adsorption include any of the naturally occurring or synthetic crystalline molecular sieves. Examples of these molecular sieves include extra-large pore zeolites, large pore zeolites, medium pore size zeolites, and small pore zeolites. These zeolites and their isotypes are classified by the Structure Commission of the International Zeolite Association according to the rules of the IUPAC Commission on Zeolite Nomenclature. According to this classification, framework type zeolites and other crystalline microporous molecular sieves, for which a structure has been established, are assigned a three letter code and are described in the “Atlas of Zeolite Framework Types”, eds. Ch. Baerlocher, L. B. McCusker, and D. H. Olson, Elsevier, Sixth Edition, 2007, which is hereby incorporated by reference. These zeolites and their isotypes are also described in http://America.iza-structure.org/IZA-SC/ftc_table.php.

The idealized inorganic framework structure of zeolites is a framework of silicate in which all tetrahedral atoms are connected by oxygen atoms with the four next-nearest tetrahedral atoms. The term “silicate”, as used herein, refers to a substance containing at least silicon and oxygen atoms that are alternately bonded to each other (i.e., —O—Si—O—Si—), and optionally including other atoms within the inorganic framework structure, including atoms such as boron, aluminum, or other metals (e.g., transition metals, such as titanium, vanadium, or zinc). Atoms other than silicon and oxygen in the framework silicate occupy a portion of the lattice sites otherwise occupied by silicon atoms in an ‘all-silica’ framework silicate. Thus, the term “framework silicate” as used herein refers to an atomic lattice comprising any of a silicate, borosilicate, gallosilicate, ferrisilicate, aluminosilicate, titanosilicate, zincosilicate, vanadosilicate, or the like.

The structure of the framework silicate within a given zeolite determines the size of the pores or channels that are present therein. The pore or channel size may determine the types of processes for which a given zeolite is applicable. Currently, greater than 200 unique zeolite framework silicate structures are known and recognized by the Structure Commission of the International Zeolite Association, thereby defining a range of pore geometries and orientations.

The framework silicates of zeolites or molecular sieves are commonly characterized in terms of their ring size, wherein the ring size refers to the number of silicon atoms (or alternative atoms, such as those listed above) that are tetrahedrally coordinated with oxygen atoms in a loop to define a pore or channel within the interior of the zeolite. For example, an “8-ring” zeolite refers to a zeolite having pores or channels defined by 8 alternating tetrahedral atoms and 8 oxygen atoms in a loop. The pores or channels defined within a given zeolite may be symmetrical or asymmetrical depending upon various structural constrains that are present in the particular framework silicate.

Zeolites can be classified as having small, medium, large, and extra-large pore structures for pore windows delimited by 8, 10, 12, and more than 12 T-atoms, respectively. Extra-large pore zeolites (>12R) include, for example, AET (14R, e.g., ALPO-8), SFN (14R, e.g., SSZ-59), VFI (18R, e.g., VPI-5), CLO (20R, cloverite), and ITV (30R, ITQ-37) framework type zeolites. Extra-large pore zeolites generally have a free pore diameter of larger than about 0.8 nm. Large pore zeolites (12R) include, for example, LTL, MAZ, FAU, EMT, OFF, *BEA, MOR, and SFE framework type zeolites, e.g., mazzite, offretite, zeolite L, zeolite Y, zeolite X, omega, ZSM-2, zeolite T, Beta, and SSZ-48. Large pore zeolites generally have a free pore diameter of 0.6 to 0.8 nm. Medium (or intermediate) pore size zeolites (10R) include, for example, MFI, MEL, EUO, MTT, MFS, AEL, AFO, HEU, FER, MWW, and TON framework type zeolites, e.g., ZSM-5, ZSM-11, ZSM-22, MCM-22, silicalite-1, and silicalite-2. Medium pore size zeolites generally have a free pore diameter of 0.45 to 0.6 nm. Small pore size zeolites (8R) include, for example, CHA, RTH, ERI, KFI, LEV, and LTA framework type zeolites, ZK-4, SAPO-34, SAPO-35, ZK-14, SAPO-42, ZK-21, ZK-22, ZK-5, ZK-20, zeolite A, chabazite, and ALPO-17. Small pore size zeolites generally have a free pore diameter of 0.3 to 0.45 nm.

Synthesis of molecular sieve materials typically involves hydrothermal crystallization from a synthesis mixture comprising sources of all the elements present in the molecular sieve (or zeolite) such as sources of silica but also of alumina etc. In many cases a structure directing agent (SDA) is also present. Structure directing agents are compounds which are believed to promote the formation of molecular sieves and which are thought to act as templates around which certain molecular sieve structures can form and which thereby promote the formation of the desired molecular sieve. Various compounds have been used as structure directing agents including various types of quaternary ammonium cations. Typically, molecular sieve (or zeolite) crystals form around structure directing agents with the structure directing agent occupying pores in the molecular sieve once crystallization is complete. The “as-synthesized” molecular sieve will therefore contain the structure directing agent in its pores so that, following crystallization, the “as-synthesized” (or “as-made”) molecular sieve is usually subjected to a treatment step such as a calcination step to remove the structure directing agent.

For instance, U.S. Pat. No. 6,080,382 and G. S. Lee et al. (2002) “Organocations in Zeolite Synthesis: Fused Bicyclo [I.m.0] Cations and the Discovery of Zeolite SSZ-48,” J. Am. Chem. Soc., v.124, pp. 7024-7034, disclose the preparation of molecular sieve SSZ-48 using a decahydroquinolinium cation as a structure directing agent, while Y. Luo et al. (2018) “A Facile and Green Method for the Synthesis of SFE Borosilicate Zeolite and Its Heteroatom-Substituted Analogues with Promising Catalytic Performances,” Chem. Eur. Jrnl., v.24(2), pp. 306-311, discloses the use of 4-dimethylaminopyridine. SSZ-48 was identified as an SFE-type zeolite that contains a one-dimensional 12-membered rings (12MR) channel system with elliptic openings (0.54×0.76 nm). SSZ-48 typically crystallizes in the form of fibrous rods having crystal dimensions of 0.05-0.25×10 microns or needles having a uniform size of about 0.05×1 microns. See also P. Wagner et al. (1999) “Electron Diffraction Structure Solution of a Nanocrystalline Zeolite at Atomic Resolution,” J. Phys. Chem. B, v.103(39), pp 8245-8250.

Although many different molecular sieves have been discovered, there is a continuing need for new molecular sieves (or zeolites) with desirable properties for gas separation and drying, organic conversion reactions, and other applications. New molecular sieves can contain novel internal pore architectures, providing enhanced selectivities in these processes. It is also important to identify new structure directing agents and more efficient methods for the synthesis of molecular sieves to facilitate the preparation of new molecular sieves and/or to reduce the cost of making known molecular sieves.

### SUMMARY

The present disclosure relates to molecular sieves, methods of making the same, and uses thereof.

In a first aspect, the present disclosure relates to a molecular sieve having, in its calcined form (e.g., where at least part of the SDA has been removed), an X-ray diffraction pattern including the following peaks in degree 2-theta in Table 1:

TABLE 1

| Degree 2-theta | Relative intensity |
|---|---|
| (±0.20) | [100 × I/(Io)] |
| 6.55 | 60-100 |
| 8.0 | 60-100 |
| 9.4 | 20-50 |
| 11.3 | 10-30 |
| 20.05 | 60-100 |
| 22.7 | 40-80 |
| 24.1 | 45-100 |
| 26.5 | 20-70 |
| 27.9 | 10-40 |

In a second aspect, the present disclosure relates to a molecular sieve having, in its as-synthesized form (e.g., where the SDA has not been removed), an X-ray diffraction pattern including the following peaks in degree 2-theta in Table 2:

TABLE 2

| Degree 2-theta | Relative intensity |
|---|---|
| (±0.20) | [100 × I/(Io)] |
| 6.55 | 10-30 |
| 8.0 | 20-50 |
| 9.4 | <20 |
| 11.3 | <20 |
| 20.05 | 30-60 |
| 22.7 | 60-100 |
| 24.1 | 60-100 |
| 26.5 | 30-60 |
| 27.9 | 20-50 |
| 35.85 | <20 |

In a third aspect, the present disclosure relates to a method of making a molecular sieve, in particular the molecular sieve of the first and/or second aspects, comprising the following steps: (a) preparing a synthesis mixture comprising water, a source of an oxide of tetravalent element (Y), optionally a source of an oxide of trivalent element (X), a structure directing agent (Q), optionally a source of hydroxide ions (OH), optionally a source of fluoride ions (F), and optionally a source of alkali and/or alkaline earth metal element (M), wherein the structure directing agent (Q) comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V:

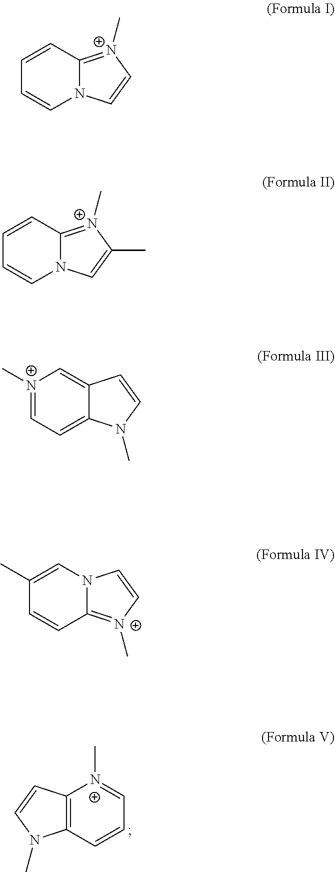

(b) heating said synthesis mixture under crystallization conditions including a temperature of from 100° C. to 200° C. for a time sufficient to form crystals of said molecular sieve; (c) recovering at least a portion of the molecular sieve from step (b); and (d) optionally treating the molecular sieve recovered in step (c) to remove at least part of the structure directing agent (Q).

In a fourth aspect, the present disclosure relates to a process of converting an organic compound to a conversion product, which comprises contacting the organic compound with the molecular sieve according to the first or second aspect of the present disclosure, or prepared according to the process of the third aspect of the present disclosure.

These and other features and attributes of the present disclosure and their advantageous applications and/or uses will be apparent from the detailed description which follows. It will of course be appreciated that features described in relation to one aspect of the present invention may be incorporated into other aspects of the present invention. In particular, any two or more of the features described in this specification, including in this summary section, can be combined to form combinations of features not specifically described herein.

### DESCRIPTION OF THE DRAWINGS

FIG. 1 shows the powder XRD pattern of the as-synthesized product of Example 2.

FIG. 2 shows the powder XRD patter of the calcined product of Example 2.

FIG. 3 shows a SEM image of the as-synthesized product of Example 2.

FIG. 4 shows SEM images of the as-synthesized product of Example 7.

FIG. 5 shows SEM images of the as-synthesized product of Example 14.

FIG. 6 shows the powder XRD pattern of the as-synthesized product of Example 17.

FIG. 7 shows the powder XRD of the calcined product of Example 17.

FIG. 8 shows a SEM image of the as-synthesized product of Example 17.

### DETAILED DESCRIPTION

The present disclosure relates to molecular sieve compositions, methods of making the same, and uses thereof. Said molecular sieves may be designated as EMM-74 molecular sieves, or EMM-74 zeolites, or EMM-74 materials.

In a first aspect, the present disclosure relates to a molecular sieve having, in its calcined form (e.g., where at least part of the SDA has been removed via thermal treatment or other treatment), an X-ray diffraction pattern including the following peaks in Table 1:

TABLE 1

| Degree 2-theta | Relative intensity |
|---|---|
| (±0.20) | [100 × I/(Io)] |
| 6.55 | 60-100 |
| 8.0 | 60-100 |
| 9.4 | 20-50 |
| 11.3 | 10-30 |
| 20.05 | 60-100 |
| 22.7 | 40-80 |
| 24.1 | 45-100 |
| 26.5 | 20-70 |
| 27.9 | 10-40 |

In a further embodiment, said molecular sieve, in its calcined form, may have an X-ray diffraction pattern including the following peaks in Table 1A, wherein the d-spacing values have a deviation determined based on the corresponding deviation ±0.20 degree 2-theta when converted to the corresponding values for d-spacing using Bragg's law:

TABLE 1A

| Degree 2-theta | d-spacing | Relative intensity |
|---|---|---|
| (±0.20) | (Å) | [100 × I/(Io)] |
| 6.55 | 13.5 | 60-100 |
| 8.0 | 11.0 | 60-100 |
| 9.4 | 9.40 | 20-50 |
| 11.3 | 7.82 | 10-30 |
| 20.05 | 4.42 | 60-100 |
| 22.7 | 3.91 | 40-80 |
| 24.1 | 3.69 | 45-100 |
| 26.5 | 3.36 | 20-70 |
| 27.9 | 3.20 | 10-40 |
| 35.85 | 2.50 | <20 |

For instance, said molecular sieve may be an aluminosilicate molecular sieve or borosilicate molecular sieve having respectively, in their calcined forms, X-ray diffraction patterns including the following peaks in Tables 1B (aluminosilicate) and 1C (borosilicate), wherein the d-spacing values have a deviation determined based on the corresponding deviation ±0.20 degree 2-theta when converted to the corresponding values for d-spacing using Bragg's law:

TABLE 1B

| Degree 2-theta | d-spacing | Relative intensity |
|---|---|---|
| (±0.20) | (Å) | [100 × I/(Io)] |
| 6.55 | 13.5 | 60-100 |
| 8.0 | 11.0 | 60-100 |
| 9.4 | 9.40 | 20-50 |
| 11.3 | 7.82 | 10-30 |
| 20.05 | 4.42 | 60-100 |
| 22.7 | 3.91 | 60-80 |
| 24.1 | 3.69 | 60-100 |
| 26.5 | 3.36 | 50-70 |
| 27.9 | 3.20 | 20-40 |
| 35.85 | 2.50 | <20 |

TABLE 1C

| Degree 2-theta | d-spacing | Relative intensity |
|---|---|---|
| (±0.20) | (Å) | [100 × I/(Io)] |
| 6.55 | 13.5 | 60-100 |
| 8.0 | 11.0 | 60-100 |
| 9.4 | 9.40 | 20-50 |
| 11.3 | 7.82 | 10-30 |
| 20.05 | 4.42 | 60-100 |
| 22.7 | 3.91 | >40-60 |
| 24.1 | 3.69 | 45-60 |
| 26.5 | 3.36 | 20-40 |
| 27.9 | 3.20 | 10-20 |
| 35.85 | 2.50 | <20 |

The XRD patterns with the XRD peaks described herein use Cu(K_{α}) radiation.

In one or more further embodiments, said molecular sieve, in its calcined form, may have a micropore volume of 0.05 to 0.25 cc/g, such as 0.07 to 0.2, e.g., 0.12 cc/g.

In one or more further embodiments, said molecular sieve, in its calcined form, may have micropore surface area of 100 to 700 m²/g, such as from 200 to 500 m²/g, e.g., 302 m²/g, and/or an external surface area of 5 to 200 m²/g, such as from 10 to 100 m²/g, e.g., 53 m²/g.

In one or more further embodiments, said molecular sieve, in its calcined form, may be optionally represented by the molecular formula of Formula V:

(m)X₂O₃:YO₂ (Formula V),

wherein 0≤m≤0.1, X is a trivalent element, and Y is a tetravalent element. Y may comprise one or more of Si, Ti, Zr, Sn, and Ge. For example, Y may comprise or be Si and/or Ge, for instance, Y may comprise or be Si. X may comprise one or more of Al, B, Fe or Ga. In particular, X may comprise or be Al and/or B, for example X may comprise or be Al. In embodiments where Y is Si and X is Al, the molecular sieve is an aluminosilicate. In embodiments where Y is Si and X is B, the molecular sieve is a borosilicate. In embodiments where Y is Si and X is a mixture of Al and B, the molecular sieve is an aluminoborosilicate. The oxygen atoms in Formula V may be replaced by carbon atoms (e.g., in the form of CH₂), which can come from sources of the components used to prepare the as-made molecular sieve.

The oxygen atoms in Formula V can also be replaced by nitrogen atoms, e.g., after the SDA has been removed. Formula V can represent the framework of a typical molecular sieve as defined in the present disclosure, in its calcined form, and is not meant to be the sole representation of said molecular sieve. Said molecular sieve, in its calcined form, may contain SDA and/or impurities after appropriate treatments to remove the SDA and impurities, which are not accounted for in Formula V. Further, Formula V does not include the protons and charge compensating ions that may be present in the calcined molecular sieve.

The variable m represents the molar ratio relationship of X₂O₃ to YO₂ in Formula V. For example, when m is 0.025, the molar ratio of YO₂ to X₂O₃ is 40 and the molar ratio of Y to X is 20 (e.g., the molar ratio of Si/Al is 20). m may vary from 0 to 0.1, such as at least 0.0017, or at least 0.005, or at least 0.007, or at least 0.01 to at most 0.1, or at most 0.07, or at most 0.05, e.g., from 0.001 or 0.005 to 0.05 or 0.025. The molar ratio of Y to X may be 5 to infinity (corresponding to a silicate product), such as at least 5, or at least 7 or at least 10, or at least 15, and up to infinity, or up to 300, or up to 100, or up to 75, or up to 50, e.g., from 10 or 15 or 20 to infinity or 300 or 100 or 75.

In a second aspect, the present disclosure relates to a molecular sieve, in particular a molecular sieve as defined in the first aspect, having in its as-synthesized form (e.g., where the SDA has not been removed) an X-ray diffraction pattern including the following peaks in degree 2-theta in Table 2:

TABLE 2

| Degree 2-theta | Relative intensity |
|---|---|
| (±0.20) | [100 × I/(Io)] |
| 6.55 | 10-30 |
| 8.0 | 20-50 |
| 9.4 | <20 |
| 11.3 | <20 |
| 20.05 | 30-60 |
| 22.7 | 60-100 |
| 24.1 | 60-100 |
| 26.5 | 30-60 |
| 27.9 | 20-50 |
| 35.85 | <20 |

In a further embodiment, said molecular sieve, in its as-synthesized form, may have an X-ray diffraction pattern including the following peaks in Table 2A, wherein the d-spacing values have a deviation determined based on the corresponding deviation ±0.20 degree 2-theta when converted to the corresponding values for d-spacing using Bragg's law:

TABLE 2A

| Degree 2-theta | d-spacing | Relative intensity |
|---|---|---|
| (±0.20) | (Å) | [100 × I/(Io)] |
| 6.55 | 13.5 | 10-30 |
| 8.0 | 11.0 | 20-50 |
| 9.4 | 9.40 | <20 |
| 11.3 | 7.82 | <20 |
| 20.05 | 4.42 | 30-60 |
| 22.7 | 3.91 | 60-100 |
| 24.1 | 3.69 | 60-100 |
| 26.5 | 3.36 | 30-60 |
| 27.9 | 3.20 | 20-50 |
| 35.85 | 2.50 | <20 |

For instance, said molecular sieve may be an aluminosilicate molecular sieve or borosilicate molecular sieve having respectively, in their as-synthesized forms, X-ray diffraction patterns including the following peaks in Tables 2B (aluminosilicate) and 2C (borosilicate), wherein the d-spacing values have a deviation determined based on the corresponding deviation ±0.20 degree 2-theta when converted to the corresponding values for d-spacing using Bragg's law:

TABLE 2B

| Degree 2-theta | d-spacing | Relative intensity |
|---|---|---|
| (±0.20) | (Å) | [100 × I/(Io)] |
| 6.55 | 13.5 | 10-30 |
| 8.0 | 11.0 | 30-50 |
| 9.4 | 9.40 | <20 |
| 11.3 | 7.82 | <20 |
| 20.05 | 4.42 | 30-60 |
| 22.7 | 3.91 | 60-100 |
| 24.1 | 3.69 | 60-100 |
| 26.5 | 3.36 | 30-60 |
| 27.9 | 3.20 | 20-50 |
| 35.85 | 2.50 | <20 |

TABLE 2C

| Degree 2-theta | d-spacing | Relative intensity |
|---|---|---|
| (±0.20) | (Å) | [100 × I/(Io)] |
| 6.55 | 13.5 | 10-30 |
| 8.0 | 11.0 | 20-40 |
| 9.4 | 9.40 | <20 |
| 11.3 | 7.82 | <20 |
| 20.05 | 4.42 | 30-60 |
| 22.7 | 3.91 | 60-100 |
| 24.1 | 3.69 | 60-100 |
| 26.5 | 3.36 | 30-60 |
| 27.9 | 3.20 | 20-50 |
| 35.85 | 2.50 | <20 |

The XRD patterns with the XRD peaks described herein use Cu(K^{α}) radiation.

In one or more further embodiments, said molecular sieve, in its as-synthesized form, may be optionally represented by the molecular formula of Formula VI:

(q)Q:(m)X₂O₃:YO₂ (Formula VI),

wherein 0<q≤0.7; 0≤m≤0.1; Q comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V:

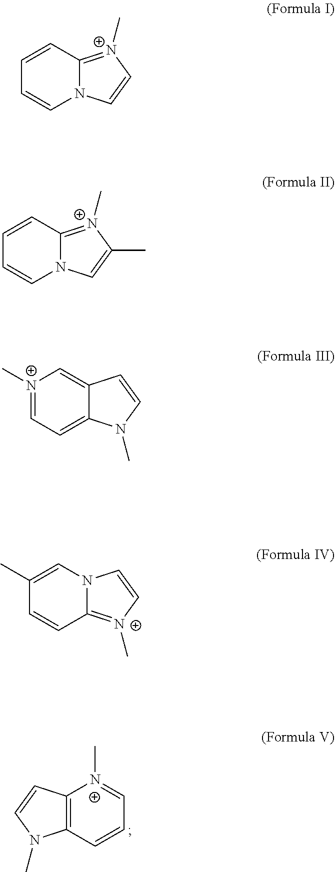

X is a trivalent element as defined for Formula V, and Y is a tetravalent element as defined for Formula V. Formula VI can represent the framework of a typical molecular sieve as defined in the present disclosure, in its as-synthesized form, therefore containing structure directing agent (Q), and is not meant to be the sole representation of such material. Said molecular sieve, in its as-synthesized form, may contain impurities which are not accounted for in Formula VI. Further, Formula VI does not include the protons and charge compensating ions that may be present in said as-synthesized molecular sieve.

The variable m represents the molar ratio relationship of X₂O₃ to YO₂ in Formula VI. The values for variable m in Formula VI are the same as those described herein for Formula V.

The variable q represents the molar relationship of Q to YO₂ in Formula VI. For example, when q is 0.1, the molar ratio of Q to YO₂ is 0.1. The molar ratio of Q to YO₂ may be from more than 0 to 0.7, such as from 0.1 to 0.6, e.g., 0.1 to 0.5.

In a further embodiment, the framework structure of the molecular sieve of the first and/or second aspects of the present disclosure (whether in as-synthesized or calcined form) may be identified as being of the SFE framework type. In particular, the framework structure of the molecular sieve of the present disclosure may be identified as possessing 12-ring pore channels (12MR) having a dimension of 5.4±0.20 Å by x 7.6±0.20 Å.

In still a further embodiment, at least a portion of the molecular sieve crystals of the present disclosure (whether in as-synthesized or calcined form) can have a rod-like morphology. By “at least a portion” of the molecular sieve crystals can have a rod-like morphology is meant at least about 50% of the molecular sieve crystals can have a rod-like morphology, such as at least 60%, at least 75%, or at least 85%. By “rod-like morphology” is meant crystals that are substantially in the form of elongated particles such as rods or cylinders, in particular with one long dimension along the c-axis (i.e., longest dimension) and one short dimension orthogonal to the long dimension (i.e., smallest dimension measured at the middle of the long dimension, perpendicular to said long dimension), where the one long dimension can be referred to as the length (1) of the crystal and the short dimension can be referred to as the width, thickness or diameter (d) of the crystal. The morphology as well as the percentage (as vol %) of crystals having said morphology can be determined by image analysis, for example, of scanning electron microscopy (SEM) micrographs, e.g., using ImageJ software.

The molecular sieve crystals having a rod-like morphology according to the present disclosure may typically have a length (1) of from 30 nm to 10 micron (μm), in particular from at least 50 nm, or at least 75 nm, or at least 100 nm, and up to at most 5 μm, or at most 1 μm, or at most 600 nm, such as from at least 100 or 150 or 200 nm and up to at most 1 μm, or 500 nm, or 400 nm, e.g., about 300 nm, 200 nm, or 100 nm, and a width, thickness or diameter (d) of from 20 nm to 200 nm, in particular from at least 25 nm, or at least 30 nm, or at least 40 nm, and up to at most 100 nm, or at most 75 nm, or at most 60 nm, such as from at least 30 or 40 or 45 nm and up to at most 100 or 75 or 60 nm, e.g., about 30 to 50 nm. The molecular sieve crystals having a rod-like morphology according to the present disclosure may typically have an aspect ratio, defined as the length to diameter ratio of a crystal (l/d), of from 1 to 10, in particular from more than 1, or at least 2, or at least 3, and up to less than 10, or at most 9, or at most 8, such as from 2 to 9, or from 3 to 8, or from 2 to 5, or from 4 to 7, e.g., about 2, 3, 4, 5 or 6.

In another further embodiment, at least a portion of the molecular sieve crystals of the present disclosure (whether in as-synthesized or calcined form) can have a plate-like morphology. By “at least a portion” of the molecular sieve crystals can have a plate-like morphology is meant at least about 50% of the molecular sieve crystals can have a plate-like morphology, such as at least 60%, at least 75%, or at least 85%. By “plate-like morphology” is meant crystals that are substantially in the form of platelets, for instance of discs or rectangular plates, having first and second major dimensions that can be referred to as the length (1) and the breadth (b) of the platelet (i.e., the longest dimension of the biggest face of the platelet and the dimension of said biggest face measured at the middle and perpendicular to said longest dimension) and a minor third dimension that can be referred to as the thickness (t) of the plate (i.e., smallest dimension measured at the middle of the longest dimension, perpendicular to said biggest face). The morphology as well as the percentage (as vol %) of crystals having said morphology can be determined by image analysis, for example, of scanning electron microscopy (SEM) micrographs, e.g., using ImageJ software.

The molecular sieve crystals having a plate-like morphology according to the present disclosure may have a length to breadth ratio (1/b) of from 1 to 5, such as from 1 to 4, or from 1 to 3, e.g., about 1, 1.5 or 2, and a length to thickness ratio (l/t) of from greater than 1 to 10, such as from 2 to 8, or from 3 to 6, e.g., about 4 or 5. The breadth to thickness ratio (b/t) may vary accordingly, for instance from greater than 1 to 10, such as from 1.5 to 8, or from 2 to 6, e.g., from 2 or 2.5 to 5. In more specific embodiments, the molecular sieve crystals having a plate-like morphology according to the present disclosure may have a length (1) of from 75 nm to 1 μm, such as at least 100 nm, and up to at most 500 nm, or at most 300 nm, or at most 200 nm, e.g., about 100 nm or 150 nm, and a thickness (t) of from 5 nm to 60 nm, such as from 10 or 15 nm to 40 or 50 nm, e.g., about 20 or 30 nm. For instance, the molecular sieve crystals may have a length (1) of about 100 to 200 nm, a breadth (b) of about 50 to 150 nm, and a thickness (t) of about 20 to 30 nm.

Rod-like and plate-like morphologies are especially advantageous in catalysis and adsorption applications as compared to fiber-like (or fibrous) morphologies that typically have an aspect ratio (l/d) higher than 10. Indeed, the diffusion rate through the longest dimension of the zeolite crystals is faster in such rod-like and plate-like particles due to their shorter diffusion length as compared to fiber-like (or fibrous) particles wherein the diffusion length is much longer. Also, fibrous crystals have produced concern as to the health effects of inhalation over a long period of time.

Therefore, in a further aspect, the present disclosure relates to a molecular sieve of SFE framework type, characterized by a rod-like or plate-like morphology, in particular by a plate-like morphology.

In a third aspect, the present disclosure relates to a method of making a molecular sieve, in particular a molecular sieve as defined in the first and/or second aspects of the present disclosure, comprising the following steps:
(a) preparing a synthesis mixture comprising water, a source of an oxide of tetravalent element (Y), optionally a source of an oxide of trivalent element (X), a structure directing agent (Q), optionally a source of hydroxide ions (OH), optionally a source of fluoride ions (F), and optionally a source of alkali and/or alkaline earth metal element (M), wherein the structure directing agent (Q) comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V:

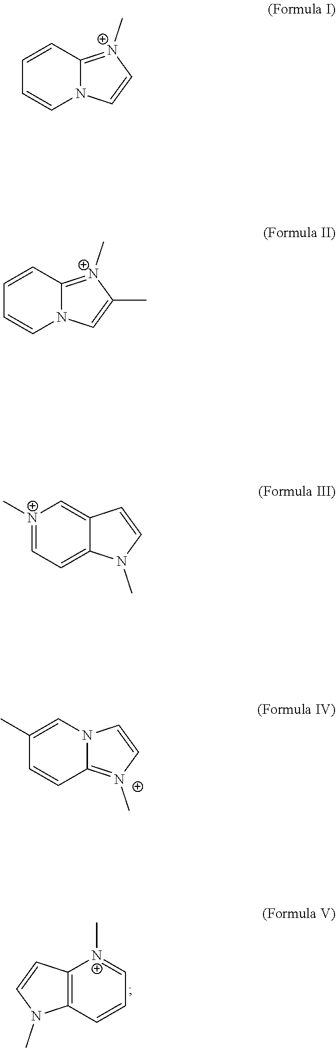

(b) heating said synthesis mixture under crystallization conditions including a temperature of from 100° C. to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

The structure directing agent (Q) may be selected from the group consisting of cations of Formula I, II and/or III as defined above, in particular it may be 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, or 1,4-dimethyl-1H-pyrrolo[3,2-b]pyridin-4-ium of Formula V. The structure directing agent (Q) may be present in any suitable form, for example as a halide, such as a fluoride, a chloride, an iodide or a bromide, as a hydroxide or as a nitrate, for instance in its hydroxide form. The structure directing agent (Q) may be present in the synthesis mixture in a Q/Y molar ratio of 0.01 to 1.0, such as 0.05 to 1.0, or at least 0.1, or at least 0.15, and up to at most 0.8, or at most 0.7, or at most 0.6, for instance 0.1 or 0.15 to 0.7 or 0.6, e.g., 0.15 to 0.5. Without wishing to be bound by theory, it has been noticed that a structure directing agent (Q) that comprises at least one cation selected from 1-methylimidazo[1,2-a]pyridin-1-ium cation of Formula I, 1,2-dimethylimidazo[1,2-a]pyridin-1-ium cation of Formula II, and 1,6-dimethylimidazo[1,2-a]pyridin-1-ium cation of Formula IV seems to favor the synthesis of molecular sieve crystals of the present disclosure having a rod-like morphology, while a structure directing agent (Q) that comprises at least one cation selected from 1,5-dimethyl-1H-pyrrolo[3,2-c]pyridin-5-ium cation of Formula III seem to favor the synthesis of molecular sieve crystals of the present disclosure having a plate-like morphology.

The synthesis mixture comprises at least one source of an oxide of tetravalent element Y which may be selected from the group consisting of Si, Ti, Zr, Sn, Ge and mixtures thereof, preferably Y comprises Si and/or Ge, e.g., Si, and more preferably Y is Si and/or Ge, e.g., Si. Suitable sources of tetravalent element Y that can be used to prepare the synthesis mixture depend on the element Y that is selected. In embodiments where Y is silicon, Si sources (e.g., silicon oxide sources) suitable for use in the method include silicates, e.g., tetraalkyl orthosilicates such as tetramethylorthosilicate (TMOS) and tetraethylorthosilicate (TEOS), fumed silica such as Aerosil® (available from Evonik), Cabosperse® (available from Cabot) and Cabosil® (available from DMS), precipitated silica such as Ultrasil® and Sipernat® 340 (available from Evonik), alkali metal silicates such as potassium silicate and sodium silicate, and aqueous colloidal suspensions of silica, for example, that sold by Grace under the tradename Ludox® or that sold by Evonik under the tradename Aerodisp®; preferably silicates, fumed silica, precipitated silica, faujasite zeolites, alkali metal silicates, and colloidal silica. In embodiments where Y is germanium, suitable Ge sources include germanium oxide. In embodiments where Y is titanium, suitable Ti sources include titanium dioxide and titanium tetraalkoxides, such as titanium (IV) tetraethoxide and titanium (IV) tetrachloride. In embodiments where Y is tin, suitable Sn sources include tin chloride and tin alkoxides, such as tin ethoxide and tin isopropoxide. In embodiments where Y is zirconium, suitable Zr sources include zirconium chloride and zirconium alkoxides, such as zirconium ethoxide and zirconium isopropoxide.

The synthesis mixture comprises at least one source of an oxide of trivalent element X which may be selected from the group consisting of Al, B, Fe, Ga, and mixtures thereof, preferably X comprises Al and/or B, e.g., Al, and more preferably X is Al and/or B, e.g., Al. Suitable sources of trivalent element X that can be used to prepare the synthesis mixture depend on the element X that is selected. In embodiments where X is aluminum, Al sources (e.g., aluminum oxide sources) suitable for use in the method include aluminum hydroxide, aluminum salts, especially water-soluble salts, such as aluminum sulfate, aluminum nitrate, alkali metal aluminates such as sodium aluminate, and aluminum alkoxides such as aluminum isopropoxide, as well as hydrated aluminum oxides, such as boehmite, gibbsite, and pseudoboehmite, and mixtures thereof. Other aluminum sources include, but are not limited to, other water-soluble aluminum salts, sodium aluminate, aluminum alkoxides, such as aluminum isopropoxide, or aluminum metal, such as aluminum in the form of chips. Especially suitable sources of alumina are aluminum hydroxide, water-soluble salts, such as aluminum sulfate, aluminum nitrate, and alkali metal aluminates such as sodium aluminate and potassium aluminate. In embodiments where X is boron, suitable B sources include boric acid and borate salts such as sodium tetraborate or borax and potassium tetraborate. Sources of boron tend to be more soluble than sources of aluminum in hydroxide-mediated synthesis systems. In embodiments where X is gallium, suitable Ga sources include sodium gallate, potassium gallate, and gallium salts such as gallium chloride, gallium sulfate, and gallium nitrate. In embodiments where X is iron, suitable Fe sources include iron chloride, iron nitrate, and iron oxides.

Alternatively, or in addition to previously mentioned sources of Y and X, sources containing both Y and X elements can also be used, such as sources of Si and Al. Examples of suitable sources containing both Si and Al elements include amorphous silica-alumina gels or dried silica alumina powders, silica aluminas, clays, such as kaolin, metakaolin, and zeolites, in particular aluminosilicates such as synthetic faujasite and ultrastable faujasite, for instance Y-type zeolite, Ultrastable Y (USY), beta or other large to medium pore zeolites.

The synthesis mixture may have a Y/X molar ratio from 5 to infinity (corresponding to a silicate product), such as at least 5 or at least 7 or at least 10, and up to infinity, or up to 300, or up to 100, or up to 75, or up to 50, for instance from 10 or 15 or 20 to infinity or 300 or 100 or 50.

In a preferred embodiment, Y is Si, optional X is Al and/or B, and the molecular sieve is a silicate, an aluminosilicate, a borosilicate, or an aluminoborosilicate. In a further preferred embodiment, Y is Si, X is Al and/or B, and the molecular sieve is an aluminosilicate, a borosilicate, or an aluminoborosilicate.

Optionally, the synthesis mixture may contain at least one source of hydroxide ions (OH). For example, hydroxide ions can be present as a counter ion of the structure directing agent (Q) or by the use of aluminum hydroxide or sodium aluminate as a source of Al. Suitable sources of hydroxide ions can also be selected from the group consisting of alkali metal hydroxides, alkaline earth metal hydroxides, ammonium hydroxide, and mixtures thereof; such as from sodium hydroxide, potassium hydroxide, lithium hydroxide, rubidium hydroxide, calcium hydroxide, magnesium hydroxide, ammonium hydroxide, and mixtures thereof; more often sodium hydroxide, potassium hydroxide, lithium hydroxide, ammonium hydroxide, and mixtures thereof; most often sodium hydroxide and/or potassium hydroxide. The synthesis mixture may comprise the hydroxide ions source in an OH/Y molar ratio of from 0 to 1.0, such as 0.05 to 0.8 or 0.1 to 0.7, or 0.15 to 0.6, e.g., from 0.25 to 0.5. Alternatively, the synthesis mixture may be substantially free from a hydroxide source.

Optionally, the synthesis mixture may comprise one or more sources of alkali or alkaline earth metal cation (M). If present, M is preferably selected from the group consisting of sodium, potassium, lithium, rubidium, calcium, magnesium and mixtures thereof, preferably sodium and/or potassium, more preferably sodium. The sodium source, when present, may be sodium hydroxide, sodium aluminate, sodium silicate, sodium aluminate or sodium salts such as NaCl, NaBr or sodium nitrate. The potassium source, when present, may be potassium hydroxide, potassium aluminate, potassium silicate, a potassium salt such as KCl or KBr or potassium nitrate. The lithium source, when present, may be lithium hydroxide or lithium salts such as LiCl, LiBr, Lil, lithium nitrate, or lithium sulfate. The rubidium source, when present, may be rubidium hydroxide or rubidium salts such as RbCl, RbBr, RBI, or rubidium nitrate. The calcium source, when present, may be calcium hydroxide, for example. The magnesium source, when present, may be magnesium hydroxide, for example. The alkali or alkaline earth metal cation M may also be present in the one or more sources of a trivalent element X, such as sodium aluminate, sodium tetraborate, potassium tetraborate, and/or in the one or more sources of tetravalent element Y, such as potassium silicate and/or sodium silicate. The synthesis mixture may comprise the alkali or alkaline earth metal cation (M) source in a M/Y molar ratio of 0 to 1.0, such as 0.05 to 0.5 or 0.05 to 0.2, e.g., from 0.1 to 0.15. Alternatively, the synthesis mixture may be substantially free from an alkali or alkaline earth metal cation (M).

The synthesis mixture may optionally comprise at least one source of fluoride ions (F). The source of fluoride ions (F) may be any compound capable of releasing fluoride ions in the molecular sieve synthesis mixture. For instance, fluoride ions can be present as a counter ion of the structure directing agent (Q). Non-limiting examples of sources of fluoride ions (F) include hydrogen fluoride (HF); salts containing one or several fluoride ions, such as metal fluoride, preferably where the metal is an alkali or alkaline earth metal such as sodium, potassium, calcium, magnesium, strontium or barium, or a metal such as aluminum (AlF₃, Al₂F₆) or tin (SnF₂); ammonium fluoride (NH₄F); and ammonium bifluoride (NH₄HF₂). Especially convenient sources of fluoride ions are HF, NH₄F, and NH₄HF₂, in particular HF. Small amounts of fluoride ions (F) may also be present as impurities, for instance in the optional source of alkali or alkaline earth metal cation (M). The fluoride ions (F) may be present in an F/Y molar ratio of 0 to 1.0, for instance 0 or 0.1 to 0.8, such as 0.2 to 0.7 or 0.3 to 0.6, e.g., 0.4 or 0.5. Alternatively, the synthesis mixture may be substantially free from fluoride ions (F).

The synthesis mixture may optionally further contain at least one source of halide ions (W), different from fluoride ions, which may be selected from the group consisting of chloride, bromide or iodide. The source of halide ions (W) may be any compound capable of releasing halide ions in the molecular sieve synthesis mixture. For instance, halide ions can be present as a counter ion of the structure directing agent (Q). Non-limiting examples of sources of halide ions include hydrogen chloride (HCl), hydrogen bromide (HBr), hydrogen iodide (HI); ammonium halides (e.g., NH₄Cl, NH₄Br, NH₄I); salts containing one or several halide ions, such as metal halides, preferably where the metal is sodium, potassium, calcium, magnesium, strontium or barium; or tetraalkylammonium halides such as tetramethylammonium halides or tetraethylammonium halides. Small amounts of halide ions (W) may also be present as impurities, for instance in the optional source of alkali or alkaline earth metal cation (M). The halide ions (W) may be present in a W/Y molar ratio of 0 to 0.2, such as 0 to 0.1, for instance less than 0.1 or even 0. In a preferred embodiment, the synthesis mixture may be substantially free from halide ions (W).

The synthesis may be performed with or without added nucleating seeds. If nucleating seeds are added to the synthesis mixture, the seeds may be of the same or of a different structure than the molecular sieve of the present disclosure, for instance EMM-74 material obtained from a previous synthesis, and may suitably present in an amount from about 0.01 ppm by weight to about 10,000 ppm by weight, based on the synthesis mixture, such as from about 100 ppm by weight to about 5,000 ppm by weight of the synthesis mixture.

The synthesis mixture typically comprises water in a H₂O/Y molar ratio of from 1 to 100, such as 5 to 80 or 7.5 to 50, for instance 7.5 or 10 to 40 or 35. Depending on the nature of the components in the base mixture, the amount of solvent (e.g., water from the hydroxide solution, and optionally methanol and ethanol from the hydrolysis of silica sources) of the base mixture may be removed such that a desired solvent to Y molar ratio is achieved for the synthesis mixture. Suitable methods for reducing the solvent content may include evaporation under a static or flowing atmosphere such as ambient air, dry nitrogen, dry air, or by spray drying or freeze drying. Water may be added to the resulting mixture to achieve a desired H₂O/Y molar ratio when too much water is removed during the solvent removal process. In some examples, water removal is not necessary when the preparation have sufficient H₂O/Y molar ratio.

Carbon in the form of CH₂ may be present in the various sources of components used to prepare the molecular sieve of the present disclosure, e.g., tetravalent element source (silica source) or trivalent element source (alumina source), and incorporated into the molecular sieve framework as bridging atoms. Nitrogen atoms may be incorporated into the framework of the molecular sieve framework as bridging atoms after the SDA has been removed.

In one or more aspects, the synthesis mixture after solvent adjustment (e.g., where the desired water to silica ratio is achieved) may be mixed by a mechanical process such as stirring or high shear blending to assure suitable homogenization of the base mixture, for example, using dual asymmetric centrifugal mixing (e.g., a FlackTek speedmixer) with a mixing speed of 1000 rpm to 3000 rpm (e.g., 2000 rpm).

The synthesis mixture is then subject to crystallization conditions suitable for the molecular sieve to form. Crystallization of the molecular sieve may be carried out under static or stirred conditions in a suitable reactor vessel, such as for example Teflon® lined or stainless steel autoclaves placed in a convection oven maintained at an appropriate temperature.

The crystallization in step (b) of the method is typically carried out at a temperature of 100° C. to 200° C., such as 120° C. to 180° C., preferably 150° C. to 170° C., for a time sufficient for crystallization to occur at the temperature used. For instance, at higher temperatures, the crystallization time may be reduced. For instance, the crystallization conditions in step (b) of the method may include heating for a period of from 1 to 100 days, such as from 1 to 50 days, for example from 1 to 30 days, e.g., at least 1 or at least 4 or at last 10 days up to 40 or 30 or 21 days. The crystallization time can be established by methods known in the art such as by sampling the synthesis mixture at various times and determining the yield and X-ray crystallinity of precipitated solid. Unless indicated otherwise herein, the temperature measured is the temperature of the surrounding environment of the material being heated, for example the temperature of the atmosphere in which the material is heated.

Typically, the molecular sieve is formed in solution and can be recovered by standard means, such as by centrifugation or filtration. The separated molecular sieve can also be washed, recovered by centrifugation or filtration and dried.

The molecular sieve of the present disclosure, when employed either as an adsorbent or as a catalyst in an organic compound conversion process may be dehydrated (e.g., dried) at least partially. This can be done by heating to a temperature in the range of 80° C. to 500° C., such as 90° C. to 370° C. in an atmosphere such as air, nitrogen, etc., and at atmospheric, subatmospheric or superatmospheric pressures for between 30 minutes and 48 hours. Dehydration may also be performed at room temperature merely by placing the molecular sieve in a vacuum, but a longer time is required to obtain a sufficient amount of dehydration.

As a result of the crystallization process, the recovered product contains within its pores at least a portion of the structure directing agent used in the synthesis. The as-synthesized molecular sieve recovered from step (c) may thus be subjected to thermal treatment or other treatment to remove part or all of the SDA incorporated into its pores during the synthesis. Thermal treatment (e.g., calcination) of the as-synthesized molecular sieve typically exposes the materials to high temperatures sufficient to remove part or all of the SDA, in an atmosphere selected from air, nitrogen, ozone or a mixture thereof in a furnace. While subatmospheric pressure may be employed for the thermal treatment, atmospheric pressure is desired for reasons of convenience. The thermal treatment may be performed at a temperature up to 925° C. e.g., 300° C. to 700° C. or 400° C. to 600° C. The temperature measured is the temperature of the surrounding environment of the sample. The thermal treatment (e.g., calcination) may be carried out in a box furnace in dry air, which has been exposed to a drying tube containing drying agents that remove water from the air. The heating is usually calcined for at least 1 minute and generally no longer than 1 or at most a few days. The heating may first be carried out under a nitrogen atmosphere and then the atmosphere may be switched to air and/or ozone.

The molecular sieve may also be subjected to an ion-exchange treatment, for example, with aqueous ammonium salts, such as ammonium nitrates, ammonium chlorides, and ammonium acetates, in order to remove remaining alkali metal cations and/or alkaline earth metal cations, if present in the synthesis mixture, and to replace them with protons thereby producing the acid form of the molecular sieve. To the extent desired, the original cations of the as-synthesized material, such as alkali metal cations, can be replaced by ion exchange with other cations. Preferred replacing cations can include hydrogen ions, hydrogen precursor, e.g., ammonium ions and mixtures thereof. The ion exchange step may take place after the as-made molecular sieve is dried. The ion-exchange step may take place either before or after a calcination step.

Optionally, aluminum atoms may be introduced in the molecular sieve framework (wherein part or all of the SDA has been removed) during an exchange process following the hydrothermal synthesis reaction. Framework silicates comprising boron atoms (e.g., borosilicates) may be particularly efficacious for undergoing exchange with aluminum atoms. Such exchange process may comprise exposing the molecular sieve to an aluminum source such as an aqueous solution comprising an aluminum salt, under conditions sufficient to exchange at least a portion and up to substantially all of the boron atoms in the framework silicate with aluminum atoms. For example, a calcined molecular sieve comprising boron may be converted to an aluminosilicate molecular sieve by heating the calcined molecular sieve comprising boron with a solution of aluminum sulfate, aluminum nitrate, aluminum chloride and/or aluminum acetate (e.g., in a sealed autoclave in a convention oven at 100° C. or at boiling temperature in an open system). The aluminum treated molecular sieve may then be recovered by filtration and washed with deionized water.

The molecular sieve may also be subjected to other treatments such as steaming and/or washing with solvent. Such treatments are well-known to the skilled person and are carried out in order to modify the properties of the molecular sieve as desired.

The molecular sieve of the present disclosure, where part or all of the SDA has been removed, may be used as an adsorbent or as a catalyst or support for catalyst in a wide variety of hydrocarbon conversions, e.g., conversion of organic compounds to a converted product. In a fourth aspect, the present disclosure therefore relates to a process of converting an organic compound to a conversion product, which comprises contacting the organic compound with the molecular sieve according to the first or second aspect of the present disclosure, or prepared according to the process of the third aspect of the present disclosure.

The molecular sieve of the present disclosure (where part or all of the SDA is removed) may be used as an adsorbent, such as for separating at least one component from a mixture of components in the vapor or liquid phase having differential sorption characteristics with respect to the material. Therefore, at least one component can be partially or substantially totally separated from a mixture of components having differential sorption characteristics with respect to the molecular sieve by contacting the mixture with said molecular sieve to selectively sorb the one component. For instance, in a process for selectively separating one or more desired components of a feedstock from remaining components of the feedstock, the feedstock may be contacted with a sorbent that comprises the molecular sieve of the present disclosure at effective sorption conditions, thereby forming a sorbed product and an effluent product. One or more of the desired components are recovered from either the sorbed product or the effluent product.

The molecular sieve of the present disclosure (where part or all of the SDA is removed) may also be used as a catalyst to catalyze a wide variety of organic compound conversion processes. Examples of chemical conversion processes, which are effectively catalyzed by the molecular sieve described herein, either alone or in combination with one or more other catalytically active substances including other crystalline catalysts, include those requiring a catalyst with acid activity. Examples of organic conversion processes, which may be catalyzed by the molecular sieve described herein include cracking, hydrocracking, isomerization, polymerisation, reforming, hydrogenation, dehydrogenation, dewaxing, hydrodewaxing, adsorption, alkylation, transalkylation, dealkylation, hydrodecylization, disproportionation, oligomerization, dehydrocyclization, conversion methanol to olefins, deNOx applications, and combinations thereof. The conversion of hydrocarbon feeds can take place in any convenient mode, for example in fluidized bed, moving bed, or fixed bed reactors depending on the types of process desired.

The molecular sieve of the present disclosure may be formulated into product compositions by combination with other materials, such as binders and/or matrix materials that provide additional hardness to the finished product. These other materials can be inert or catalytically active materials.

For instance, it may be desirable to incorporate the molecular sieve of the present disclosure with another material that is resistant to the temperatures and other conditions employed during use. Such materials include synthetic or naturally occurring zeolites as well as inorganic materials such as clays, silica and/or metal oxides such as alumina and mixtures thereof. The metal oxides may be either naturally occurring or in the form of gelatinous precipitates or gels including mixtures of silica and metal oxides. Use of a resistant material in conjunction with the molecular sieve of the present disclosure, i.e., combined therewith or present during synthesis of the as-made molecular sieve, which crystal is active, tends to change the conversion and/or selectivity of the catalyst in certain organic conversion processes. Inactive resistant materials suitably serve as diluents to control the amount of conversion in a given process so that products can be obtained in an economic and orderly manner without employing other means for controlling the rate of reaction. These materials may be incorporated into naturally occurring clays, e.g., bentonite and kaolin, to improve the crush strength of the product under commercial operating conditions. Said inactive resistant materials, i.e., clays, oxides, etc., function as binders for the catalyst. A catalyst having good crush strength can be beneficial because in commercial use, it is desirable to prevent the catalyst from breaking down into powder-like materials.

Naturally occurring clays which may be used include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or after being subjected to calcination, acid treatment or chemical modification. Binders useful for compositing with the molecular sieve of the present disclosure also include inorganic oxides selected from silica, zirconia, titania, magnesia, beryllia, alumina, yttria, gallium oxide, zinc oxide and mixtures thereof.

In addition to the foregoing materials, the molecular sieve of the present disclosure may be composited with a porous matrix material such as silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

These binder materials are resistant to the temperatures and other conditions, e.g., mechanical attrition, which occur in various hydrocarbon separation processes. Thus the molecular sieve of the present disclosure may be used in the form of an extrudate with a binder. They are typically bound by forming a pill, sphere, or extrudate. The extrudate is usually formed by extruding the molecular sieve, optionally in the presence of a binder, and drying and calcining the resulting extrudate. Further treatments such as steaming, and/or ion exchange may be carried out as required. The molecular sieve may optionally be bound with a binder having a surface area of at least 100 m²/g, for instance at least 200 m²/g, optionally at least 300 m²/g.

The relative proportions of molecular sieve and inorganic oxide matrix may vary widely, with the molecular sieve content ranging from about 1 to about 100 percent by weight and more usually, particularly when the composite is prepared in the form of extrudates, in the range of about 2 to about 95, optionally from about 20 to about 90 weight percent of the composite.

The molecular sieve of the present disclosure may also be used in intimate combination with a hydrogenating component such as tungsten, vanadium, molybdenum, rhenium, nickel, cobalt, chromium, manganese, or a noble metal such as platinum or palladium where a hydrogenation-dehydrogenation function is to be performed. Such hydrogenating components may be incorporated in the composition by way of one or more of the following processes: cocrystallization; exchanged into the composition to the extent a Group IIIA element, e.g., aluminium, is in the structure; or intimately physically admixed therewith. Such components can also be impregnated in or onto the molecular sieve, for example, by treating the molecular sieve with a hydrogenating metal-containing ion. For instance, in the case of platinum, suitable platinum compounds for this purpose include chloroplatinic acid, platinous chloride and various compounds containing a platinum amine complex. Combinations of metals and methods for their introduction can also be used.

It will be understood by a person skilled in the art that the molecular sieve of the present disclosure may contain impurities, such as amorphous materials, unit cells having different topologies (e.g., quartz or molecular sieves of different framework type, that may or may not impact the performance of the resulting catalyst), and/or other impurities (e.g., heavy metals and/or organic hydrocarbons). Typical examples of molecular sieves of different framework type co-existing with the molecular sieve of the present disclosure are e.g., molecular sieves of ferrierite or FER framework type, such as ZSM-35. The molecular sieve of the present disclosure is preferably substantially free of impurities. The term “substantially free of impurities” (or in the alternative “substantially pure”) used herein means the molecular sieve contains a minor proportion (less than 50 wt %), preferably less than 20 wt %, more preferably less than 10 wt %, even more preferably less than 5 wt % and most preferably less than 1 wt % (e.g., less than 0.5 wt % or 0.1 wt %), of such impurities (e.g., “non-EMM-74 material”), which weight percent (wt %) values are based on the combined weight of impurities and pure molecular sieve. The amount of impurities can be appropriately determined by powder XRD, rotating electron diffraction, and/or SEM/TEM (e.g., different crystal morphologies).

The molecular sieve described herein is substantially crystalline. As used herein, the term “crystalline” refers to a crystalline solid form of a material, including, but not limited to, a single-component or multiple-component crystal form, e.g., including solvates, hydrates, and a co-crystal. Crystalline can mean having a regularly repeating and/or ordered arrangement of molecules and possessing a distinguishable crystal lattice. For example, the molecular sieve can have different water or solvent content. The different crystalline lattices can be identified by solid state characterization methods such as by XRD (e.g., powder XRD). Other characterization methods known to a person of ordinary skill in the relevant art can further help identify the crystalline form as well as help determine stability and solvent/water content. As used herein, the term “substantially crystalline” means a majority (greater than 50 wt %) of the weight of a sample of a material described is crystalline and the remainder of the sample is a non-crystalline form. In one or more aspects, a substantially crystalline sample has at least 95% crystallinity (e.g., 5% of the non-crystalline form), at least 96% crystallinity (e.g., 4% of the non-crystalline form), at least 97% crystallinity (e.g., 3% of the non-crystalline form), at least 98% crystallinity (e.g., about 2% of the non-crystalline form), at least 99% crystallinity (e.g., 1% of the non-crystalline form), and 100% crystallinity (e.g., 0% of the non-crystalline form).

Aspects of the disclosure are described in greater detail by way of specific examples. The following examples are offered for illustrative purposes and are not intended to limit the disclosure in any manner. Those of skill in the relevant art will readily recognize a variety of parameters can be changed or modified to yield essentially the same results.

### EXAMPLES

The present invention is further illustrated below without limiting the scope thereto.

In these examples, the X-ray diffraction (XRD) patterns of the as-synthesized and calcined materials were recorded on an X-Ray Powder Diffractometer (Bruker DaVinci D8 Discovery instrument) in continuous mode using a Cu Kα radiation, Bragg-Bentano geometry with Vantec 500 detector, in the 2θ range of 2 to 50 degrees. The interplanar spacings, d-spacings, were calculated in Angstrom units, and the relative intensities of the lines, I/Iₒ is the ratio of the peak intensity to that of the intensity of the strongest line, above background. The intensities are uncorrected for Lorentz and polarization effects. The location of the diffraction peaks in 2-theta, and the relative peak area intensities of the lines, I/I(o), where Io is the intensity of the strongest line, above background, were determined with the MDI Jade peak search algorithm. It should be understood that diffraction data listed as single lines may consist of multiple overlapping lines which under certain conditions, such as differences in crystallographic changes, may appear as resolved or partially resolved lines. Typically, crystallographic changes can include minor changes in unit cell parameters and/or a change in crystal symmetry, without a change in the framework connectivity. These minor effects, including changes in relative intensities, can also occur as a result of differences in cation content, framework composition, nature and degree of pore filling, crystal size and shape, preferred orientation and thermal and/or hydrothermal history.

The scanning electron microscopy (SEM) images of the as-synthesized materials were obtained on a Hitachi 4800 Scanning Electron Microscope. SEM images were used to aid assessment of product purity. The presence of obviously different crystal morphologies in a SEM image can be an indication of impurities in the form of other crystalline materials. Such an approximate analysis can be especially useful in identifying the presence of formation of relatively minor amounts of crystalline impurities which may not be identifiable on product XRD patterns.

The overall BET surface area (S_{BET}) of the materials was determined by the BET method as described by S. Brunauer, et al. (1938) “Adsorption of Gases in Multimolecular Layers,” J. Am. Chem. Soc., v.60(2), pp. 309-319, incorporated herein by reference, using nitrogen adsorption-desorption at liquid nitrogen temperature. The external surface area (Sₑₓₜ) of the material was obtained from the t-plot method, and the micropore surface area (S_{micro}) of the material was calculated by subtracting the external surface area (Sₑₓₜ) from the overall BET surface area (S_{BET}).

The micropore volume (V_{micro}) and total pore volume (Vₜₒₜ) of the materials can be determined using methods known in the relevant art. For example, the micropore and total pore volumes of the materials can be measured with nitrogen physisorption, and the data can be analyzed by the t-plot method described in Lippens, B. C. et al. (1965) “Studies on Pore System in Catalysts: V. The t Method”, J. Catal., v.4(3), pp. 319-323, which describes micropore volume and total pore volume methods and is incorporated herein by reference.

The molar ratios and conditions used for the syntheses of Examples 2-14, as well as the resulting products, are detailed below and summarized in Table 3.

### Example 1a: Synthesis of 1-methylimidazo[1,2-a]pyridin-1-ium cation (SDA-1)

1-methylimidazo[1,2-a]pyridin-1-ium iodide: A mixture of 25 g (211.61 mmol) of imidazo[1,2-a]pyridine and 69.08 g (486.71 mmol) of methyl iodide in 265 mL of acetonitrile was heated to 80° C. for 10 hours. Thin layer chromatography showed remaining starting material after 10 hours. The reaction mixture was cooled to room temperature and the acetonitrile removed via rotovap. The resulting yellow oil was triturated with 200 mL of ethyl acetate and a yellow solid collected. ¹H-NMR showed the presence of the desired product and an impurity. The impurity was removed by heating the yellow solid to 50° C. in 200 mL of acetone and filtering the remaining solid material. The ¹H-NMR of the filtered material confirmed the clean desired product had been obtained.

1-methylimidazo[1,2-a]pyridin-1-ium hydroxide: The iodide salt was ion-exchanged with ion-exchange resin Amberlite® IRN78 OH hydroxide form (with iodide:resin:water ratio of 1:3.5:5) to the hydroxide form. The exchange was performed at room temperature overnight.

### Example 1b: Synthesis of 1,2-dimethylimidazo[1,2-a]pyridin-1-ium cation (SDA-2)

1,2-dimethylimidazo[1,2-a]pyridin-1-ium iodide: A mixture of 26.52 g (200.65 mmol) of 2-methylimidazo[1,2-a]pyridine and 29.90 g (210.68 mmol) of methyl iodide in 250 mL of acetonitrile was stirred at room temperature for 16 hours. The solids were filtered and rinsed with 200 mL of ethyl acetate and allowed to air dry for 30 minutes prior to taking an ¹H-NMR. The ¹H-NMR confirmed the clean desired product had been obtained.

1,2-dimethylimidazo[1,2-a]pyridin-1-ium hydroxide: The iodide salt was ion-exchanged with ion-exchange resin Amberlite® IRN78 OH hydroxide form (with iodide:resin:water ratio of 1:3.5:5) to the hydroxide form. The exchange was performed at room temperature overnight.

### Example 1c: Synthesis of 1,5-dimethy-1H-pyrrolo[3,2-c]pyridin-5-ium cation (SDA-3)

1,5-dimethy-1H-pyrrolo[3,2-c]pyridin-5-ium iodide: 25.0 g (221.6 mmol) of 1H-pyrrolo[3,2-c]pyridine, 69.1 g (486.7 mmol) of methyl iodide, and 13.1 g (232.8 mmol) of potassium hydroxide in 400 mL of acetonitrile were heated to 80° C. for 6 hours. The acetonitrile was removed via rotavap and the residue dissolved in 250 mL of chloroform. The potassium salts were removed by filtration and subsequently the chloroform was removed via rotavap. The residue was triturated with 350 mL of ethyl acetate and the product precipitated out as a mustardy colored solid. The solid was collected and allowed to air dry for 30 minutes to give 55.5 g (96%) of 1,5-dimethy-1H-pyrrolo[3,2-c]pyridin-5-ium iodide, as confirmed by ¹H-NMR.

1,5-dimethy-1H-pyrrolo[3,2-c]pyridin-5-ium hydroxide: The iodide salt was ion-exchanged with ion-exchange resin Amberlite® IRN78 OH hydroxide form (with iodide:resin:water ratio of 1:3.5:5) to the hydroxide form. The exchange was performed at room temperature overnight.

### Example 1d: Synthesis of 1,6-dimethylimidazo[1,2-a]pyridin-1-ium cation (SDA-4)

1,6-dimethylimidazo[1,2-a]pyridin-1-ium iodide: To a stirred solution of 25 g (189.15 mmol) of 6-methylimidazo[1,2-a]pyridine in 236 mL of acetonitrile was added 53.7 g (378.3 mmol) of methyl iodide dropwise, at room temperature. The reaction was monitored by thin layer chromatography (TLC). Upon disappearance of the starting 6-methylimidazo[1,2-a]pyridine, ether was added and a solid formed. The solid was collected via filtration and rinsed with 300 mL of ether and allowed to air dry. The reaction yielded 439 g of the desired product, as confirmed by ¹H-NMR.

1,6-dimethylimidazo[1,2-a]pyridin-1-ium hydroxide: The iodide salt was ion-exchanged with ion-exchange resin Amberlite® IRN78 OH hydroxide form (with iodide:resin:water ratio of 1:3.5:5) to the hydroxide form. The exchange was performed at room temperature overnight.

### Example 1e: Synthesis of 1,4-dimethy-1H-pyrrolo[3,2-b]pyridin-4-ium cation (SDA-5)

1,4-dimethy-1H-pyrrolo[3,2-b]pyridin-4-ium iodide: 18.7 g of 4-azaindole, 52 g of methyl iodide, and 10.0 g of potassium hydroxide in 200 mL of acetonitrile were heated to 70° C. overnight. The acetonitrile was removed via rotavap and the residue dissolved in chloroform. The potassium salts were removed by filtration and subsequently the chloroform was removed via rotavap. H-NMR showed that the brown solid 1,4-dimethy-1H-pyrrolo[3,2-b]pyridin-4-ium iodide (or 1,4-dimethyl-4-azaindolium iodide).

1,4-dimethy-1H-pyrrolo[3,2-b]pyridin-4-ium hydroxide: The iodide salt was ion-exchanged with ion-exchange resin Amberlite® IRN78 OH hydroxide form (with iodide:resin:water ratio of 1:3.5:5) to its hydroxide form. The exchange was performed at room temperature overnight.

### Example 2: SDA-2, USY Zeolite, NaOH, Si/Al=15

In a PTFE liner for a 10 mL Steel Parr autoclave, the following were mixed together: 2.7 g of 1,2-dimethylimidazo[1,2-a]pyridin-1-ium (SDA-2) hydroxide solution (5.9 wt %), 0.96 g of NaOH solution (4 wt %), and 0.46 g of Ultrastable Y (USY) zeolite with a Si/Al molar ratio of 15 (available from Zeolyst as CBV720) to produce a synthesis mixture having the following composition in terms of molar ratios:

30 H₂O: 1 SiO₂: 0.033 Al₂O₃: 0.15 QOH: 0.15 NaOH.

The liner was then capped, sealed within a 10 mL Parr autoclave, and placed within a spit inside of a convection oven. The reactor was heated at 160° C. for 1 week under tumbling conditions (about 30 rpm). The product was isolated by filtration, rinsed with deionized water and dried at 90° C. in a vented drying oven. The as-synthesized material was then calcined to 580° C. in air within a box furnace with a ramping rate of 3° C./minute. The temperature remained at 580° C. for 8 hours and then the box furnace was allowed to cool.

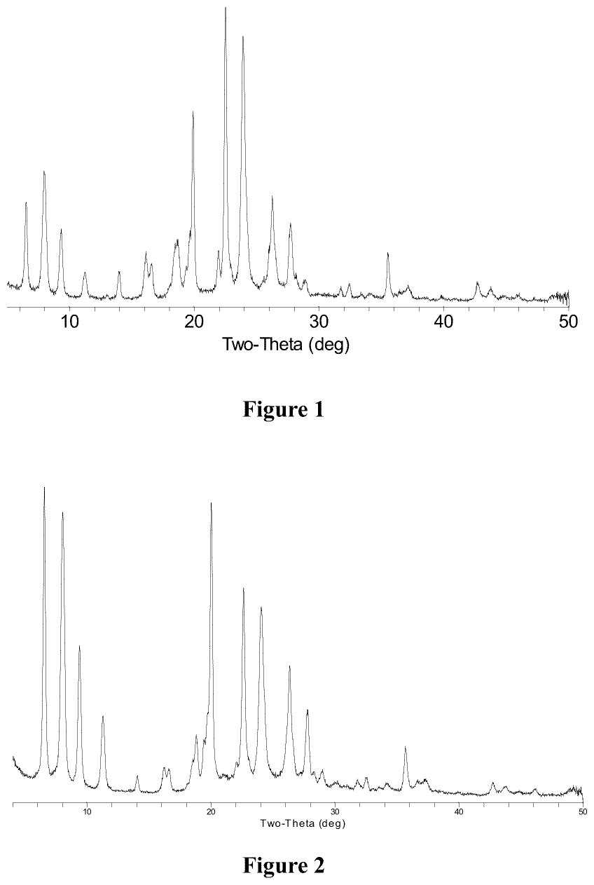

XRD analysis of the as-synthesized and calcined products showed the material to have similar powder XRD patterns to respectively as-synthesized and calcined SSZ-48 molecular sieve in terms of degree 2-theta and d-spacing but with different relative intensities (as illustrated in Tables I and II of, e.g., U.S. Pat. No. 6,080,382, which is incorporated herein by reference in its entirety). This new product was identified as EMM-74. FIGS. 1 and 2 show the powder XRD patterns of the as-synthesized and calcined EMM-74 materials of Example 2. Tables 4 and 5 below show the list of peaks and intensities for the as-synthesized and calcined EMM-74 materials or Example 2.

EMM-74 material was shown to have a SFE framework type with 12-ring pore channels (12MR) having a dimension of 5.4×7.6 Å.

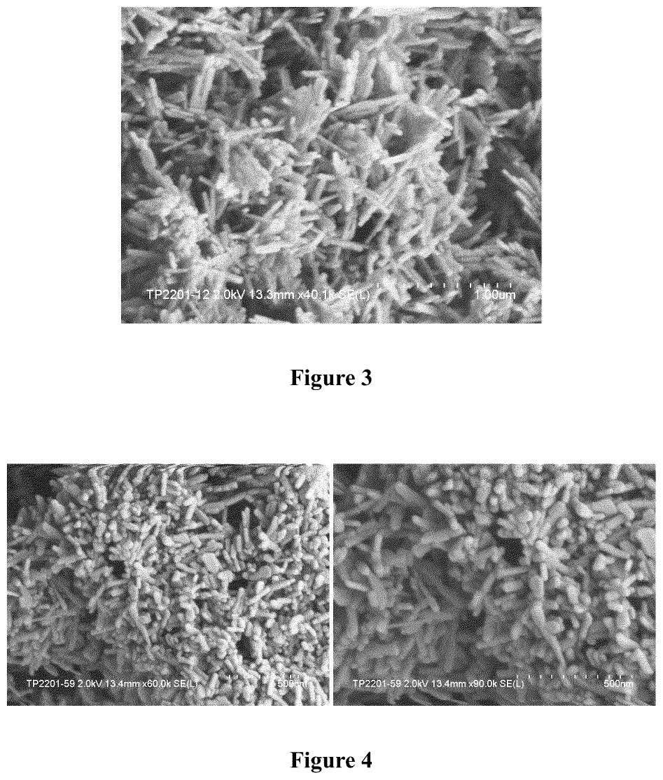

EMM-74 material had a uniform rod-like morphology with a particle diffusion length of about 300 nm, a diameter (or width or thickness) of about 50 nm, and an aspect ratio (l/d) of about 6, as illustrated by FIG. 3 which shows a SEM image of the as-synthesized product of Example 2.

The calcined EMM-74 material had a micropore surface area (S_{micro}) of 302 m²/g, an external surface area (Sₑₓₜ) of 53 m²/g, a total pore volume (Vₜₒₜ) of 0.31 cm³/g and a micropore volume (V_{micro}) of 0.12 cm³/g. The adsorption uptake for n-hexane, 2,2-dimethylbutane, 2,3-dimethylbutane, and mesitylene were 73 mg/g, 67 mg/g, 57 mg/g, and 59 mg/g, respectively. These data demonstrate that much of the micropore volume in EMM-74 is available to these adsorbate molecules. The 12MR pore channels are evidently large enough to accommodate 1,3,5-trimethylbenzene (mesitylene), leading to similar capacity of the smaller absorbates.

### Example 3: SDA-2, USY Zeolite, Sodium Aluminate, NaOH, Si/Al=16.5

This Example was conducted in similar conditions as Example 2 except that the Ultrastable Y (USY) zeolite with a Si/Al molar ratio of 30 (available from Zeolyst as CBV760) and sodium aluminate (NaAlO₂, 25 wt % Al₂O₃, 19.3 wt % Na₂O) were used as the Si and Al sources, resulting in the following composition in terms of molar ratios:

30 H₂O:1SiO₂:0.03Al₂O₃:0.15QOH:0.15NaOH.

After 10 days of heating at 160° C., pure EMM-74 product was obtained, as identified by its XRD pattern. Said EMM-74 product had a rod-like morphology similar to the product of Example 2, as determined by SEM.

### Example 4: SDA-2, USY Zeolite, Sodium Aluminate, NaOH, Si/Al=25

This Example was conducted in similar conditions as Example 3 except that the synthesis mixture contained a lower amount of Al and a lower amount of NaOH, resulting in the following composition in terms of molar ratios:

30 H₂O:1SiO₂:0.02Al₂O₃:0.15QOH:0.1NaOH.

After 10 days of heating at 160° C., pure EMM-74 product was obtained, as identified by its XRD pattern. Said EMM-74 product had a rod-like morphology similar to the product of Example 2, as determined by SEM.

### Example 5: SDA-2, Colloidal Silica, Sodium Aluminate, NaOH, Si/Al=15

This Example was conducted in similar conditions as Example 3 except that Ludox HS40 (40 wt % colloidal silica suspension) was used as the Si source and sodium aluminate (NaAlO₂, 25 wt % Al₂O₃, 19.3 wt % Na₂O) was used as the Al source, to give a synthesis mixture having the following composition in terms of molar ratios:

33 H₂O:1SiOd2:0.033Al₂O₃:0.15QOH:0.15NaOH.

After 3 weeks of heating at 160° C., EMM-74 product with a minor amount of FER material was obtained, as identified by its XRD pattern. Said EMM-74 product had a rod-like morphology similar to the product of Example 2, as determined by SEM.

### Example 6: SDA-2, Colloidal Silica, Sodium Aluminate, NaOH, Si/Al=22.5

This Example was conducted in similar conditions as Example 5 except that the synthesis mixture contained a lower amount of Al and of NaOH, resulting in a synthesis mixture having the following composition in terms of molar ratios:

33 H₂O:1SiO₂:0.022Al₂O₃:0.15QOH:0.10NaOH.

After 3 weeks of heating at 160° C., EMM-74 product with a minor amount of amorphous phase material was obtained, as identified by its XRD pattern. Said EMM-74 product had a rod-like morphology similar to the product of Example 2, as determined by SEM.

### Example 7: SDA-2, USY Zeolite, KOH, Si/Al=15

This Example was conducted in similar conditions as Example 2 except that NaOH was replaced by KOH (20%), the synthesis mixture also containing a lower amount of water, resulting in a synthesis mixture having the following composition in terms of molar ratios:

22 H₂O:1SiO₂:0.033Al₂O₃:0.15QOH:0.15KOH.

After 1 week of heating at 160° C., pure EMM-74 product was obtained, as identified by its XRD pattern.

The EMM-74 material of example 7 had a uniform rod-like morphology with a particle diffusion length of about 50-200 nm, a diameter (or width or thickness) of about 20-50 nm, and an aspect ratio (l/d) of about 2-8, as illustrated by FIG. 4 which shows a SEM image of the as-synthesized product of Example 7.

### Example 8: SDA-2, USY Zeolite, KOH, Si/Al=30

This Example was conducted in similar conditions as Example 7 except that Ultrastable Y (USY) zeolite with a Si/Al molar ratio of 30 (available from Zeolyst as CBV760) was used as the Si and Al sources, the synthesis mixture also containing a higher amount of water and a lower amount of KOH, resulting in the following composition in terms of molar ratios:

30 H₂O:1SiO₂:0.017Al₂O₃:0.15QOH:0.1KOH.

After 2 weeks of heating at 160° C., pure EMM-74 product was obtained, as identified by its XRD pattern. Said EMM-74 product had a rod-like morphology similar to the product of Example 2, as determined by SEM.

### Example 9: SDA-2, USY Zeolite, KOH, Si/Al=40

This Example was conducted in similar conditions as Example 8 except that the synthesis mixture contained a lower amount of Al, Ultrastable Y (USY) zeolite with a Si/Al molar ratio of 40 (available from Zeolyst as CBV780) being used as the Si and Al sources, resulting in the following composition in terms of molar ratios:

30 H₂O:1SiO₂:0.0125Al₂O₃:0.15QOH:0.1KOH.

After 2 weeks of heating at 160° C., EMM-74 product with a minor amount of quartz was obtained, as identified by its XRD pattern. Said EMM-74 product had a rod-like morphology similar to the product of Example 2, as determined by SEM.

### Example 10: SDA-2, TEOS, Aluminum Hydroxide, HF, Si/Al=25

1.43 g of tetraethylorthosilicate (TEOS, >99 wt %) and 0.026 g of aluminum hydroxide (Sigma, 54 wt % Al₂O₃) were hydrolyzed at room temperature in 9.6 g of 1,2-dimethylimidazo[1,2-a]pyridin-1-ium (SDA-2) hydroxide solution (5.9 wt %) for about 2 to 3 hours. The mixture was then heated at about 50° C. to remove the ethanol and water. Subsequently, 0.14 HF (48 wt % solution) were added to the mixture to produce a synthesis mixture having the following composition in terms of molar ratios:

7.5 H₂O:1SiO₂:0.02Al₂O₃:0.5QOH:0.5HF.

The resulting thick paste was homogenized by hand and transferred in a PTFE liner for a 10 mL Steel Parr autoclave. The reactor was heated at 160° C. for 3 week under tumbling conditions (about 40 rpm). The product was isolated by centrifugation, washing three times with distilled water (100 mL) and dried at 90° C. in a vented drying oven. The as-synthesized material was then calcined to 580° C. in air within a box furnace with a ramping rate of 3° C./minute. The temperature remained at 580° C. for 8 hours and then the box furnace was allowed to cool.

XRD analysis of the as-synthesized product showed the material to be pure EMM-74 product. Said EMM-74 product had a rod-like morphology similar to the product of Example 2, as determined by SEM.

### Example 11: SDA-2, TEOS, Aluminum Hydroxide, HF, Si/A1=50

This Example was conducted in similar conditions as Example 10 except that the synthesis mixture contained a lower amount of Al and a higher amount of water, resulting in the following composition in terms of molar ratios:

15 H₂O:1SiO₂:0.01Al₂O₃:0.5QOH:0.5HF.

After 3 weeks of heating at 160° C., EMM-74 product with a minor amount of an unidentified impurity was obtained, as identified by its XRD pattern. Said EMM-74 product had a rod-like morphology similar to the product of Example 2, as determined by SEM.

### Example 12: SDA-1, TEOS, Al Hydroxide, HF, Si/Al=50

This Example was conducted in the same conditions as Example 11 except that 1-methylimidazo[1,2-a]pyridin-1-ium (SDA-1) hydroxide was used rather than SDA-2 hydroxide. The synthesis mixture had the following composition in terms of molar ratios: 15 H₂O:1 SiO₂: 0.01 Al₂O₃: 0.5 QOH: 0.5 HF.

After 10 days of heating at 160° C., pure EMM-74 product was obtained, as identified by its XRD pattern. Said EMM-74 product had a rod-like morphology similar to the product of Example 2, as determined by SEM.

### Example 13: SDA-4, USY Zeolite, NaOH, Si/Al=15

Similarly to the procedure of Example 2, in a PTFE liner for a 10 mL Steel Parr autoclave, the following were mixed together: 1.41 g of 1,6-dimethylimidazo[1,2-a]pyridin-1-ium (SDA-4) hydroxide solution (15 wt %), 0.51 g of NaOH solution (10 wt %), 2.9 g of deionized water, and 0.61 g of Ultrastable Y (USY) zeolite with a Si/Al molar ratio of 15 (available from Zeolyst as CBV720) to produce a synthesis mixture having the following composition in terms of molar ratios:

30 H₂O:1SiO₂:0.033Al₂O₃:0.15QOH:0.15NaOH.

The liner was then capped, sealed within a 10 mL Parr autoclave, and placed within a spit inside of a convection oven under tumbling conditions (about 30 rpm). After 20 days of heating at 160° C., pure EMM-74 product was obtained, as identified by its XRD pattern. Said EMM-74 product had a rod-like morphology similar to the product of Example 2, as determined by SEM.

### Example 14: SDA-3, USY Zeolite, NaOH, Si/Al=15

Similarly to the procedure of Example 2, in a PTFE liner for a 10 mL Steel Parr autoclave, the following were mixed together: 3.8 g of 1,5-dimethy-1H-pyrrolo[3,2-c]pyridin-5-ium (SDA-3) hydroxide solution (5.5 wt %), 0.51 g of NaOH solution (10 wt %), and 0.61 g of Ultrastable Y (USY) zeolite with a Si/Al molar ratio of 15 (available from Zeolyst as CBV720) to produce a synthesis mixture having the following composition in terms of molar ratios:

30 H₂O:1SiO₂:0.033Al₂O₃:0.15QOH:0.15NaOH.

The liner was then capped, sealed within a 10 mL Parr autoclave, and placed within a spit inside of a convection oven. The reactor was heated at 160° C. for 10 days under tumbling conditions (about 30 rpm). The product was isolated by filtration, rinsed with deionized water and dried at 90° C. in a vented drying oven. The as-synthesized material was then calcined to 580° C. in air within a box furnace with a ramping rate of 3° C./minute. The temperature remained at 580° C. for 8 hours and then the box furnace was allowed to cool.

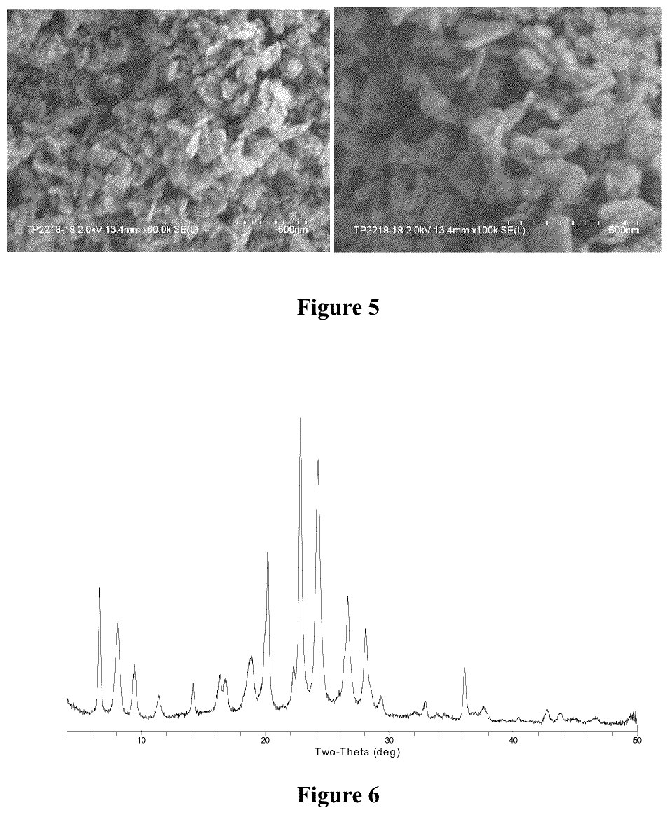

XRD analysis of the as-synthesized product showed the material to be pure EMM-74 product. As illustrated by FIG. 5 which shows SEM images of the as-synthesized product of Example 14, the EMM-74 aluminosilicate of Example 14, prepared using SDA-3, has a uniform plate-like morphology with a length of about 100-150 nm, a breadth of about 50-100, and a thickness of about 20-30 nm.

### Example 15: SDA-3, USY Zeolite, NaOH, Si/Al=15

Example 14 was reproduced with a slightly lower amount of water, resulting in a synthesis mixture having the following composition in terms of molar ratios:

27 H₂O:1SiO₂:0.033Al₂O₃:0.15QOH:0.15NaOH.

After 10 days of heating at 170° C., pure EMM-74 product was obtained, as identified by its XRD pattern. Said EMM-74 product has a plate-like morphology similar to the product of Example 14, as determined by SEM.

### Example 16: SDA-3, Colloidal Silica, Aluminum Hydroxide, NaOH, Si/Al=20

This Example was conducted in similar conditions as Example 14 except that Ludox AS40 (40 wt % colloidal silica suspension) was used as the Si source and aluminum hydroxide (Sigma, 54 wt % Al₂O₃) was used as the Al source, to give a synthesis mixture having the following composition in terms of molar ratios:

30 H₂O:1SiO₂:0.025Al₂O₃:0.15QOH:0.15NaOH.

After 10 days of heating at 160° C., EMM-74 product with a minor amount of FER material was obtained, as identified by its XRD pattern. Said EMM-74 product has a plate-like morphology similar to the product of Example 14, as determined by SEM.

### Example 17: SDA-2, colloidal silica, boric acid, Si/B=10

In a PTFE liner for a 10 mL Steel Parr autoclave, the following were mixed together: 8.6 g of 1,2-dimethylimidazo[1,2-a]pyridin-1-ium (SDA-2) hydroxide solution (5.9 wt %), 1.16 g of Ludox HS40 (40 wt % colloidal silica suspension), and 1.23 g of boric acid (3.87 wt % H₃BO₃ solution), the water was allowed to vaporize to produce a synthesis mixture having the following composition in terms of molar ratios:

10 H₂O:1SiO₂:0.05B₂O₃:0.4QOH.

The liner was then capped, sealed within a 10 mL Parr autoclave, and placed within a spit inside of a convection oven. The reactor was heated at 160° C. for 3 weeks under tumbling conditions (about 30 rpm). The product was isolated by filtration, rinsed with deionized water and dried at 90° C. in a vented drying oven. The as-synthesized material was then calcined to 580° C. in air within a box furnace with a ramping rate of 3° C./minute. The temperature remained at 580° C. for 8 hours and then the box furnace was allowed to cool.

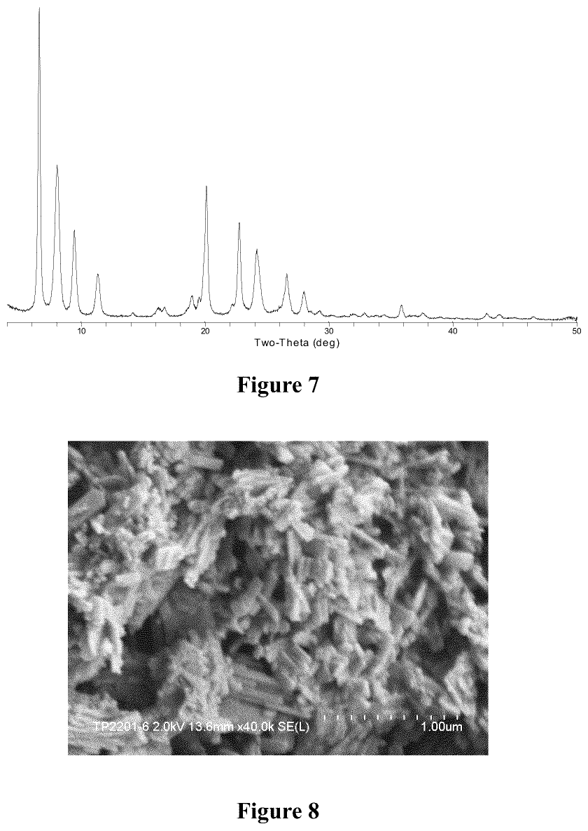

FIGS. 6 and 7 show the powder XRD patterns of the as-synthesized and calcined materials, corresponding to pure EMM-74 products. Tables 6 and 7 below show the list of peaks and intensities for the as-synthesized and calcined EMM-74 materials of Example 17.

As illustrated by FIG. 8 which shows a SEM image of the as-synthesized product of Example 17, the EMM-74 borosilicate of Example 17 had a uniform rod-like morphology similar to the product of Example 2, with a particle diffusion length of about 300 nm and a diameter (or width or thickness) of about 50 nm.

### Example 18: SDA-2, Y-type Zeolite, boric acid, NaOH, Si/B=16.5, Si/Al=50

This Example was conducted in similar conditions as Example 3 except that Y-type zeolite with a Si/Al molar ratio of 50 (available from Tosoh as HSZ-385HUA) was used as a source of Si and Al, and boric acid was added as a source of B (3.87 wt % H₃BO₃ solution), resulting in the following composition in terms of molar ratios:

30 H₂O:1SiO₂:0.03B₂O₃:0.01Al₂O₃:0.15QOH:0.15NaOH.

After 10 days of heating at 160° C., a pure aluminoborosilicate EMM-74 product was obtained, as identified by its XRD pattern. Said EMM-74 product had a rod-like morphology similar to the product of Example 2, as determined by SEM.

### Example 19: SDA-2, Y-Type Zeolite, Boric Acid, NaOH, Si/B=10, Si/Al=250

This Example was conducted in similar conditions as Example 18 except that the synthesis mixture contained a lower amount of water, a higher amount of boron, and a lower amount of aluminum, Y-type zeolite with a Si/Al molar ratio of 250 (available from Tosoh as HSZ-390HUA) being used as the source of Si and Al, resulting in the following composition in terms of molar ratios:

25 H₂O:1SiO₂:0.05B₂O₃:0.002Al₂O₃:0.15QOH:0.15NaOH.

After 6 days of heating at 160° C., pure EMM-74 product was obtained, as identified by its XRD pattern. Said EMM-74 product had a rod-like morphology similar to the product of Example 2, as determined by SEM.

### Example 20: SDA-5, USY Zeolite, KOH, Si/Al=15

In a PTFE liner for a 23 mL Steel Parr autoclave, the following were mixed together: 3.1 g of 1,4-dimethy-1H-pyrrolo[3,2-b]pyridin-4-ium (SDA-5) hydroxide solution (9.4 wt %), 0.36 g of KOH solution (20 wt %), 2.25 g of deionized water and 0.61 g of Ultrastable Y (USY) zeolite with a Si/Al molar ratio of 15 (available from Zeolyst as CBV720) to produce a synthesis mixture having the following composition in terms of molar ratios:

30 H₂O:1SiO₂:0.033Al₂O₃:0.15QOH:0.15KOH.

The liner was then capped, sealed within a 23 mL Parr autoclave, and placed within a spit inside of a convection oven. The reactor was heated at 160° C. for 8 days under tumbling conditions (about 30 rpm). The product was isolated by filtration, rinsed with deionized water and dried.

XRD analysis of the as-synthesized product showed the material to be pure EMM-74 product.

TABLE 3

| Ex. | Si Source | X Source | SDA* | Si/X | H₂O/Si | Q/Si | OH/Si | M/Si | T (° C.) | t | | Product |
|---|---|---|---|---|---|---|---|---|---|---|---|---|
| 2 | USY | USY | SDA-2 | 15 | 30 | 0.15 | 0.30 | 0.15 (Na) | 160 | 1 | week | EMM-74 |
| | (Si/Al = 15) | (Si/Al = 15) | | | | | | | | | | |
| 3 | USY | NaAlO₂ | SDA-2 | 16.5 | 30 | 0.15 | 0.30 | 0.15 (Na) | 160 | 10 | days | EMM-74 |
| | (Si/Al = 30) | | | | | | | | | | | |
| 4 | USY | NaAlO₂ | SDA-2 | 25 | 30 | 0.15 | 0.25 | 0.1 (Na) | 160 | 10 | days | EMM-74 |
| | (Si/Al = 30) | | | | | | | | | | | |
| 5 | Ludox HS40 | NaAlO₂ | SDA-2 | 15 | 33 | 0.15 | 0.30 | 0.15 (Na) | 160 | 3 | weeks | EMM-74 + |
| | | | | | | | | | | | | FER |
| 6 | Ludox HS40 | NaAlO₂ | SDA-2 | 22.5 | 33 | 0.15 | 0.25 | 0.1 (Na) | 160 | 3 | weeks | EMM-74 + |
| | | | | | | | | | | | | amorphous |
| | | | | | | | | | | | | phase |
| 7 | USY | USY | SDA-2 | 15 | 22 | 0.15 | 0.30 | 0.15 (K) | 160 | 1 | week | EMM-74 |
| | (Si/Al = 15) | (Si/Al = 15) | | | | | | | | | | |
| 8 | USY | USY | SDA-2 | 30 | 30 | 0.15 | 0.25 | 0.1 (K) | 160 | 2 | weeks | EMM-74 |
| | (Si/Al = 30) | (Si/Al = 30) | | | | | | | | | | |
| 9 | USY | USY | SDA-2 | 40 | 30 | 0.15 | 0.25 | 0.1 (K) | 160 | 2 | weeks | EMM-74 + |
| | (Si/Al = 40) | (Si/Al = 40) | | | | | | | | | | quartz |
| 10 | TEOS | Al(OH)₃ | SDA-2 | 25 | 7.5 | 0.50 | 0.50 | 0 | 160 | 3 | weeks | EMM-74 |
| 11 | TEOS | Al(OH)₃ | SDA-2 | 50 | 15 | 0.50 | 0.50 | 0 | 160 | 3 | weeks | EMM-74 + |
| | | | | | | | | | | | | impurity |
| 12 | TEOS | Al(OH)₃ | SDA-1 | 50 | 15 | 0.5 | 0.5 | 0 | 160 | 10 | days | EMM-74 |
| 13 | USY | USY | SDA-4 | 15 | 30 | 0.15 | 0.30 | 0.15 (Na) | 160 | 20 | days | EMM-74 |
| | (Si/Al = 15) | (Si/Al = 15) | | | | | | | | | | |
| 14 | USY | USY | SDA-3 | 15 | 30 | 0.15 | 0.30 | 0.15 (Na) | 160 | 10 | days | EMM-74 |
| | (Si/Al = 15) | (Si/Al = 15) | | | | | | | | | | |
| 15 | USY | USY | SDA-3 | 15 | 27 | 0.15 | 0.30 | 0.15 (Na) | 170 | 10 | days | EMM-74 |
| | (Si/Al = 15) | (Si/Al = 15) | | | | | | | | | | |
| 16 | Ludox AS40 | Al(OH)₃ | SDA-3 | 20 | 30 | 0.15 | 0.30 | 0.15 (Na) | 160 | 10 | days | EMM-74 |
| 17 | Ludox HS40 | H₃BO₃ | SDA-2 | 10 | 10 | 0.40 | 0.40 | 0 | 160 | 3 | weeks | EMM-74 |
| 18 | Y-Type Zeolite | H₃BO₃ | SDA-2 | 16.5 (Si/B) | 30 | 0.15 | 0.30 | 0.15 (Na) | 170 | 10 | days | EMM-74 |
| | (Si/Al = 50) | | | 50 (Si/Al) | | | | | | | | |
| 19 | Y-Type Zeolite | H₃BO₃ | SDA-2 | 10 (Si/B) | 25 | 0.15 | 0.30 | 0.15 (Na) | 160 | 6 | days | EMM-74 |
| | (Si/Al = 250) | | | 250 (Si/Al) | | | | | | | | |
| 20 | USY | USY | SDA-5 | 15 | 30 | 0.15 | 0.30 | 0.15 (K) | 160 | 8 | days | EMM-74 |
| | (Si/Al = 15) | (Si/Al = 15) | | | | | | | | | | |

*SDA-1 = 1-methylimidazo[1,2-a]pyridin-1-ium cation
SDA-2 = 1,2-dimethylimidazo[1,2-a]pyridin-1-ium cation
SDA-3 = 1,5-dimethy-1H-pyrrolo[3,2-c]pyridin-5-ium cation
SDA-4 = 1,6-dimethylimidazo[1,2-a]pyridin-1-ium cation
SDA-5 = 1,4-dimethy-1H-pyrrolo[3,2-b]pyridin-4-ium cation

TABLE 4

| Degree 2-theta | d-spacing | Relative intensity |
|---|---|---|
| (±0.20) | (Å) | [100 × I/(Io)] |
| 6.53 | 13.52 | 21.7 |
| 7.99 | 11.05 | 36.7 |
| 9.31 | 9.49 | 14.1 |
| 11.24 | 7.87 | 4.9 |
| 13.97 | 6.33 | 4.6 |
| 16.12 | 5.49 | 8.6 |
| 16.52 | 5.36 | 9.2 |
| 18.66 | 4.75 | 20.3 |
| 19.93 | 4.45 | 45.5 |
| 22.50 | 3.95 | 73 |
| 23.88 | 3.72 | 100 |
| 26.23 | 3.39 | 44.1 |
| 27.62 | 3.23 | 27.5 |
| 28.89 | 3.09 | 3.2 |
| 35.48 | 2.53 | 8.7 |

TABLE 5

| Degree 2-theta | d-spacing | Relative intensity |
|---|---|---|
| (±0.20) | (Å) | [100 × I/(Io)] |
| 6.56 | 13.47 | 76 |
| 8.03 | 11.00 | 98.6 |
| 9.38 | 9.42 | 37 |
| 11.26 | 7.85 | 19.8 |
| 7.53 | 11.74 | 1.3 |
| 16.23 | 5.46 | 7.3 |
| 18.77 | 4.72 | 8.7 |
| 18.83 | 4.71 | 14.3 |
| 20.03 | 4.43 | 90.1 |
| 22.61 | 3.93 | 71.7 |
| 24.01 | 3.70 | 100 |
| 26.32 | 3.38 | 62 |
| 27.73 | 3.21 | 3 |
| 28.96 | 3.08 | 7 |
| 35.62 | 2.52 | 14.8 |

TABLE 6

| Degree 2-theta | d-spacing | Relative intensity |
|---|---|---|
| (±0.20) | (Å) | [100 × I/(Io)] |
| 6.62 | 13.33 | 20 |
| 8.09 | 10.92 | 28.5 |
| 9.44 | 9.37 | 10.2 |
| 11.36 | 7.78 | 3.9 |
| 14.16 | 6.25 | 6 |
| 16.30 | 5.43 | 13.5 |
| 16.78 | 5.28 | 8.1 |
| 18.86 | 4.70 | 25.3 |
| 20.20 | 4.39 | 50.9 |
| 22.83 | 3.89 | 76.2 |
| 24.21 | 3.67 | 100 |
| 26.65 | 3.34 | 49.5 |
| 28.04 | 3.18 | 33 |
| 29.30 | 3.05 | 3.4 |
| 36.00 | 2.49 | 10.5 |

TABLE 7

| Degree 2-theta | d-spacing | Relative intensity |
|---|---|---|
| (±0.20) | (Å) | [100 × I/(Io)] |
| 6.59 | 13.40 | 100 |
| 8.05 | 10.98 | 96.5 |
| 9.42 | 9.38 | 40 |
| 11.32 | 7.81 | 21.5 |
| 14.25 | 6.21 | 0.7 |
| 16.26 | 5.45 | 4.2 |
| 16.66 | 5.32 | 2.8 |
| 18.92 | 4.69 | 9.3 |
| 20.10 | 4.41 | 66.1 |
| 22.73 | 3.91 | 45.2 |
| 24.15 | 3.68 | 49 |
| 26.59 | 3.35 | 28.9 |
| 27.93 | 3.19 | 13.7 |
| 29.25 | 3.05 | 2.4 |
| 35.80 | 2.51 | 3.5 |

While the present invention has been described and illustrated with reference to particular embodiments, it will be appreciated by those of ordinary skill in the art that the invention lends itself to many different alterations, modifications, and variations not specifically illustrated herein. It will also be apparent to those skilled in the art that when numerical lower limits and numerical upper limits are listed herein, ranges from any lower limit to any upper limit are contemplated. Also, all numerical values within the detailed description herein are modified by “about” the indicated value, and take into account experimental error and variations that would be expected by a person having ordinary skill in the art.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

Additionally or alternately, the invention relates to:

Embodiment 1: A molecular sieve having, in its calcined form, an X-ray diffraction pattern including the peaks in Table 1.

Embodiment 2: The molecular sieve of embodiment 1 having a molecular formula of Formula V:

(m)X₂O₃:YO₂ (Formula V),

wherein 0≤m≤0.1, X is a trivalent element, and Y is a tetravalent element.

Embodiment 3: The molecular sieve of embodiment 2, wherein X comprises one or more of aluminum, boron, iron, and gallium, preferably X comprises or is aluminum and/or boron.

Embodiment 4: The molecular sieve of embodiment 2 or 3, wherein Y comprises one or more of silicon, titanium, zirconium, tin and germanium, preferably Y comprises or is silicon and/or germanium, more preferably silicon.

Embodiment 5: A molecular sieve having, in its as-synthesized form, an X-ray diffraction pattern including the peaks in Table 2.

Embodiment 6: The molecular sieve of embodiment 5 having a molecular formula of Formula V:

(q)Q:(m)X₂O₃:YO₂ (Formula V),

wherein 0<q≤0.7, 0≤m≤0.1, X is a trivalent element, Y is a tetravalent element, and Q comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V:

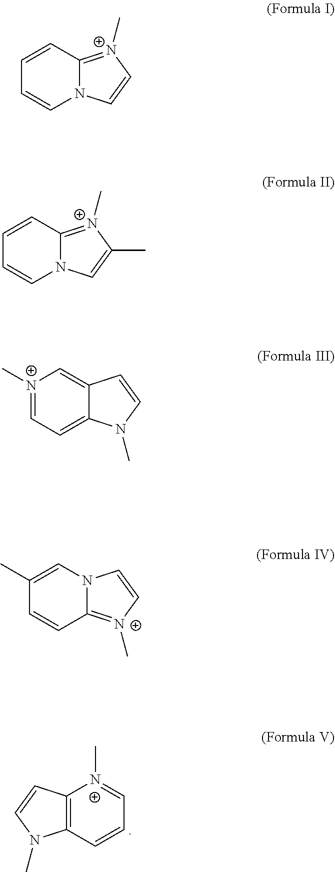

Embodiment 7: The molecular sieve of embodiment 6, wherein X comprises one or more of aluminum, boron, iron, and gallium, preferably X comprises or is aluminum and/or boron.

Embodiment 8: The molecular sieve of embodiment 6 or 7, wherein Y comprises one or more of silicon, titanium, zirconium, tin and germanium, preferably Y comprises or is silicon and/or germanium, more preferably silicon.

Embodiment 9: The molecular sieve of any one of the preceding embodiments, wherein at least a portion of the molecular sieve crystals have a rod-like morphology.

Embodiment 10: The molecular sieve of embodiment 9, having a length of from 30 nm to 10 mm, preferably of from 100 nm to 1 mm, more preferably of from 100 nm to 500 nm, and an aspect ratio of from more than 1 to 10, in particular from 2 to 8.

Embodiment 11: The molecular sieve of any one of embodiments 1 to 8, wherein at least a portion of the molecular sieve crystals have a plate-like morphology.

Embodiment 12: The molecular sieve of embodiment 11, having a length of from 75 nm to 1 mm, preferably of from 75 nm to 500 nm, more preferably of from 100 nm to 300 nm, and a thickness of from 5 nm to 60 nm, in particular from 10 nm to 50 nm, more particularly from 20 to 30 nm.

Embodiment 13: The molecular sieve of any one of the preceding embodiments, which is a silicate, an aluminosilicate, a borosilicate, or an aluminoborosilicate and which has a Si/X molar ratio of from 5 to 300 (if Al and/or B is present), preferably from 10 to 100, more preferably from 15 to 75.

Embodiment 14: A method of making the molecular sieve of any one of the preceding embodiments, comprising:
(a) preparing a synthesis mixture comprising water, a source of an oxide of tetravalent element (Y), a source of an oxide of trivalent element (X), a structure directing agent (Q), optionally a source of hydroxide ions (OH), optionally a source of fluoride ions (F), and optionally a source of alkali and/or alkaline earth metal element (M),
wherein the structure directing agent (Q) comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V:

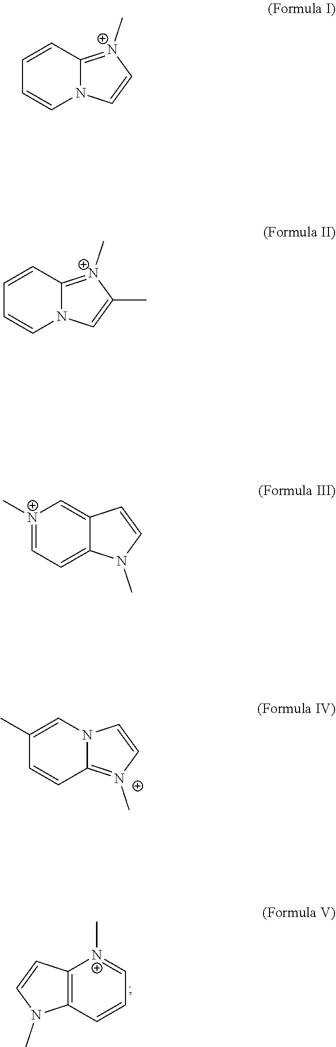

(b) heating said synthesis mixture under crystallization conditions including a temperature of from 100° C. to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

Embodiment 15: The method of embodiment 14, wherein the structure directing agent (Q) is in the form of a halide, hydroxide or nitrate, preferably wherein the structure directing agent (Q) is in its hydroxide form.

Embodiment 16: The method of embodiment 14 or 15, wherein the tetravalent element (Y) is selected from the group consisting of silicon, titanium, zirconium, tin, germanium, and mixtures thereof, preferably wherein the tetravalent element (Y) comprises silicon and/or germanium, more preferably wherein the tetravalent element (Y) is silicon and/or germanium, most preferably silicon.

Embodiment 17: The method of any one of embodiments 14 to 16, wherein the trivalent element (X) is selected from the group consisting of aluminum, boron, iron, gallium, and mixtures thereof, preferably wherein the trivalent element (X) comprises aluminum and/or boron, more preferably wherein the trivalent element (X) is aluminum and/or boron, in particular aluminum.

Embodiment 18: The method of any one of embodiments 14 to 17, wherein the synthesis mixture has the following composition in terms of molar ratios:

| Molar ratios | Typical range | Preferred range | More preferred range |
|---|---|---|---|
| Y/X | 5-∞ | 5-300 (if X present) | 10-100 (if X present) |
| Q/Y | 0.05-1.0 | 0.1-0.8 | 0.15-0.6 |
| OH/Y | 0-1.0 | 0.05-0.8 (if OH present) | 0.15-0.6 (if OH present) |
| M/Y | 0-1.0 | 0.05-0.5 (if M present) | 0.05-0.2 (if M present) |
| F/Y | 0-1.0 | 0.1-0.8 (if F present) | 0.2-0.7 (if F present) |
| H₂O/Y | 1-100 | 5-80 | 10-50 |

Embodiment 19: A process of converting an organic compound to a conversion product comprises contacting the organic compound with the molecular sieve of any one of embodiments 1 to 13.

1. A molecular sieve having, in its calcined form, an X-ray diffraction pattern including the following peaks in Table 1:
TABLE 1

| Degree 2-theta | Relative intensity |
|---|---|
| (±0.20) | [100 × I/(Io)] |
| 6.55 | 60-100 |
| 8.0 | 60-100 |
| 9.4 | 20-50 |
| 11.3 | 10-30 |
| 20.05 | 60-100 |
| 22.7 | 40-80 |
| 24.1 | 45-100 |
| 26.5 | 20-70 |
| 27.9 | 10-40 |

2. The molecular sieve of claim 1 having a molecular formula of Formula V:
(m)X₂O₃:YO₂ (Formula V),
wherein 0≤m≤0.1, X is a trivalent element, and Y is a tetravalent element; in particular wherein X comprises one or more of aluminum, boron, iron, and gallium, and Y comprises one or more of silicon, titanium, zirconium, tin and germanium.

3. A molecular sieve having, in its as-synthesized form, an X-ray diffraction pattern including the following peaks in Table 2:
TABLE 2

| Degree 2-theta | Relative intensity |
|---|---|
| (±0.20) | [100 × I/(Io)] |
| 6.55 | 10-30 |
| 8.0 | 20-50 |
| 9.4 | <20 |
| 11.3 | <20 |
| 20.05 | 30-60 |
| 22.7 | 60-100 |
| 24.1 | 60-100 |
| 26.5 | 30-60 |
| 27.9 | 20-50 |
| 35.85 | <20 |

4. The molecular sieve of claim 3 having a molecular formula of Formula VI:
(q)Q:(m)X₂O₃:YO₂ (Formula VI),
wherein 0<q≤0.7, 0≤m≤0.1, X is a trivalent element, Y is a tetravalent element, and Q comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V: 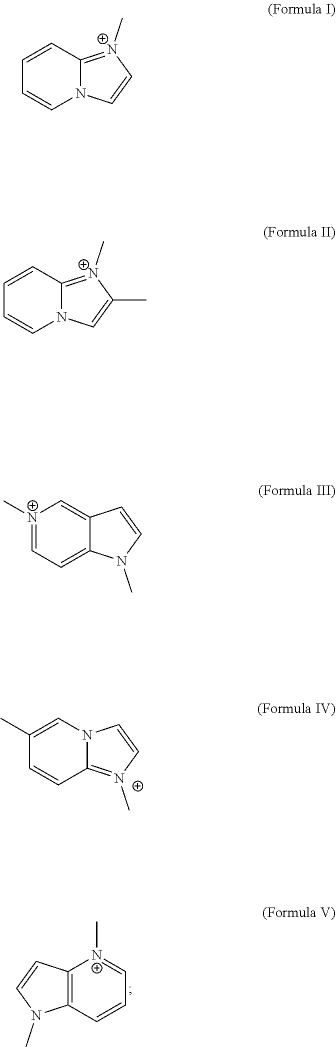
in particular, wherein X comprises one or more of aluminum, boron, iron, and gallium, and Y comprises one or more of silicon, titanium, zirconium, tin and germanium.

5. The molecular sieve of claim 1, wherein at least a portion of the molecular sieve crystals have a rod-like morphology.

6. The molecular sieve of claim 5, having a length of from 30 nm to 10 mm, and an aspect ratio of from more than 1 to 10.

7. The molecular sieve of claim 1, wherein at least a portion of the molecular sieve crystals have a plate-like morphology.

8. The molecular sieve of claim 1, which is a silicate, an aluminosilicate, a borosilicate, or an aluminoborosilicate and which has a Si/X molar ratio of from 5 to 300 (if Al and/or B is present).

9. A method of making the molecular sieve of claim 1, comprising:
(a) preparing a synthesis mixture comprising water, a source of an oxide of tetravalent element (Y), a source of an oxide of trivalent element (X), a structure directing agent (Q), optionally a source of hydroxide ions (OH), optionally a source of fluoride ions (F), and optionally a source of alkali and/or alkaline earth metal element (M),
wherein the structure directing agent (Q) comprises at least one cation selected from 1-methylimidazo[1,2-a]pyridin-1-ium cation of Formula I, 1,2-dimethylimidazo[1,2-a]pyridin-1-ium cation of Formula II, 1,5-dimethyl-1H-pyrrolo[3,2-c]pyridin-5-ium cation of Formula III, 1,6-dimethylimidazo[1,2-a]pyridin-1-ium cation of Formula IV, and 1,4-dimethyl-1H-pyrrolo[3,2-b]pyridin-4-ium of Formula V: 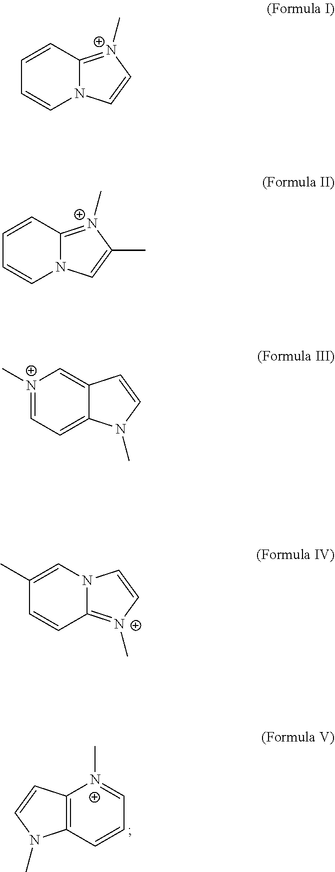
(b) heating said synthesis mixture under crystallization conditions including a temperature of from 100° C. to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

10. The method of claim 9, wherein the structure directing agent (Q) is in the form of a halide, hydroxide or nitrate.

11. The method of claim 9, wherein the tetravalent element (Y) is selected from the group consisting of silicon, titanium, zirconium, tin, germanium, and mixtures thereof.

12. The method of claim 9, wherein the trivalent element (X) is selected from the group consisting of aluminum, boron, iron, gallium, and mixtures thereof.

13. The method of claim 9, wherein the synthesis mixture has the following composition in terms of molar ratios:

| Molar ratios | ranges |
|---|---|
| Y/X | 5-∞ |
| Q/Y | 0.05-1.0 |
| OH/Y | 0-1.0 |
| M/Y | 0-1.0 |
| F/Y | 0-1.0 |
| H₂O/Y | 1-100 |

14. A process of converting an organic compound to a conversion product comprises contacting the organic compound with the molecular sieve of claim 1.
